(19) United States
(12) Patent Application Publication
(10) Pub. No.: US 2025/0107731 A1
(43) Pub. Date: Apr. 3, 2025

(54) ENDOSCOPE SYSTEM, IMAGE PROCESSING APPARATUS, OPERATION METHOD OF ENDOSCOPE SYSTEM, AND NON-TRANSITORY COMPUTER READABLE MEDIUM

(71) Applicant: FUJIFILM Corporation, Tokyo (JP)

(72) Inventor: Takahiro OKAMOTO, Kanagawa (JP)

(73) Assignee: FUJIFILM Corporation, Tokyo (JP)

(21) Appl. No.: 18/898,683

(22) Filed: Sep. 27, 2024

(30) Foreign Application Priority Data
Sep. 28, 2023 (JP) 2023-168896

(51) Int. Cl. A61B 5/1459; A61B 1/00; A61B 1/04; A61B 5/00; A61B 5/1455

(52) U.S. Cl. CPC A61B 5/1459; A61B 1/00009; A61B 1/00045; A61B 1/04; A61B 5/14551; A61B 5/7278

(57) ABSTRACT

The endoscope system that calculates the oxygen saturation acquires an image obtained by imaging an observation target, sets a plurality of regions of interest in the image, performs determination processing of determining, with respect to each of the plurality of regions of interest, whether or not correction of data using the region of interest is possible, and corrects the data using the region of interest for which it is determined that the correction of the data is possible.

### CROSS-REFERENCE TO RELATED APPLICATIONS

This application claims priority under 35 U.S.C. § 119(a) to Japanese Patent Application No. 2023-168896 filed on 28 Sep. 2023. The above application is hereby expressly incorporated by reference, in its entirety, into the present application.

### BACKGROUND OF THE INVENTION

### 1. Field of the Invention

The present invention relates to an endoscope system, an image processing apparatus, an operation method of an endoscope system, and a non-transitory computer readable medium.

### 2. Description of the Related Art

In recent years, in medical fields using an endoscope, a technique of obtaining a biological parameter, such as oxygen saturation, using an image obtained by imaging an observation target with an endoscope (hereinafter, referred to as an endoscope image) is known. Oxygen saturation imaging is a technique of generating an image representing oxygen saturation of hemoglobin including the observation target (hereinafter, referred to as an oxygen saturation image) using an endoscope image.

In the oxygen saturation imaging, in order to accurately calculate the oxygen saturation of the observation target, data used in calculating the oxygen saturation is corrected using the endoscope image. A case can occur where during correction, correction of the data used in calculating the oxygen saturation of the observation target fails due to halation occurring in the endoscope image, residues or residual liquids adhering to the observation target, or the like. An endoscope system that determines whether correction is successful or unsuccessful is known (JP2017-080246A, corresponding to US2018/235439A1). In addition, an endoscope system that has a correction mode in which a specific coloring agent concentration is calculated from a plurality of image signals and correction processing of data used in calculating oxygen saturation is performed is known (WO2023/119856A).

### SUMMARY OF THE INVENTION

Examples of one effective method of accurately calculating the oxygen saturation of the observation target include a method of appropriately correcting the data used in calculating the oxygen saturation. In addition, since the correction is often performed during an endoscope operation, it is preferable to quickly perform the correction.

An object of the present invention is to provide an endoscope system, an image processing apparatus, an operation method of an endoscope system, and a non-transitory computer readable medium that can more reliably and quickly correct data used in calculating oxygen saturation.

According to an aspect of the present invention, there is provided an endoscope system that calculates oxygen saturation of an observation target using data used in calculating the oxygen saturation, the endoscope system comprising a processor, in which the processor is configured to acquire an image obtained by imaging the observation target, set a plurality of regions of interest in the image, perform determination processing of determining, with respect to each of the plurality of regions of interest, whether or not correction of the data using the region of interest is possible, and correct the data using the region of interest for which it is determined that the correction of the data is possible.

It is preferable that the determination processing includes region reliability degree calculation processing of calculating a region reliability degree of each of the plurality of regions of interest based on the region of interest and representative region determination processing of determining a representative region by comparing the region reliability degrees of the plurality of respective regions of interest with each other.

It is preferable that the region reliability degree is calculated using a pixel reliability degree calculated for each pixel included in each of the plurality of regions of interest.

It is preferable that the determination processing includes region reliability degree comparison processing of comparing the region reliability degree of the representative region determined through the representative region determination processing with a region reliability degree threshold value set in advance.

It is preferable that the processor is configured to correct the pixel reliability degree according to a position of each pixel in the image. It is preferable that the region reliability degree is calculated using the corrected pixel reliability degree.

It is preferable that in the region reliability degree calculation processing, the region reliability degree is calculated after performing weighting according to an area of the region of interest.

It is preferable that in the region reliability degree calculation processing, the region reliability degree is calculated after performing weighting according to a distance of the region of interest from a center of the image.

It is preferable that the processor is configured to set the regions of interest such that each of the plurality of regions of interest has substantially the same shape.

It is preferable that the processor is configured to set the regions of interest such that each of the plurality of regions of interest has substantially the same area.

It is preferable that the processor is configured to set the regions of interest that have shapes different from each other and/or areas different from each other based on positions of the regions of interest in the image.

It is preferable that the processor is configured to set the regions of interest such that at least two of the plurality of regions of interest include the same pixel of the image.

It is preferable that the processor is configured to set the regions of interest such that each of the plurality of regions of interest includes pixels of the image different from each other.

It is preferable that the processor is configured to perform control of giving a notification to a user in a case where there is no region of interest for which it is determined that the correction of the data is possible.

It is preferable that the processor is configured to perform control of displaying the region of interest for which it is determined that the correction of the data is possible on a display.

It is preferable that the processor is configured to perform control of displaying a pixel having a pixel reliability degree equal to or higher than a pixel reliability degree threshold value set in advance on a display in an aspect of being distinguishable from other pixels.

It is preferable that the processor is configured to calculate, in a case where the data is corrected, the oxygen saturation of the observation target using the corrected data.

It is preferable that the processor is configured to calculate, in a case where there is no region of interest for which it is determined that the correction of the data is possible, the oxygen saturation of the observation target using the uncorrected data.

It is preferable that the processor is configured to correct the data using image information of the region of interest for which it is determined that the correction of the data is possible.

It is preferable that the image information is a specific coloring agent concentration of a specific coloring agent other than blood hemoglobin.

It is preferable that the specific coloring agent is a yellow coloring agent.

According to another aspect of the present invention, there is provided an image processing apparatus that calculates oxygen saturation of an observation target, the image processing apparatus comprising a processor, in which the processor is configured to acquire an image obtained by imaging the observation target, set a plurality of regions of interest in the image, perform determination processing of determining, with respect to each of the plurality of regions of interest, whether or not correction of data using the region of interest is possible, and correct the data using the region of interest for which it is determined that the correction of the data is possible.

According to still another aspect of the present invention, there is provided an operation method of an endoscope system of calculating oxygen saturation of an observation target, the operation method comprising a step of acquiring an image obtained by imaging the observation target, a step of setting a plurality of regions of interest in the image, a step of performing determination processing of determining, with respect to each of the plurality of regions of interest, whether or not correction of data using the region of interest is possible, and a step of correcting the data using the region of interest for which it is determined that the correction of the data is possible.

According to still another aspect of the present invention, there is provided a non-transitory computer readable medium for storing a computer-executable program for causing a computer to function as an endoscope system that calculates oxygen saturation of an observation target, the computer-executable program causing the computer to realize a function of acquiring an image obtained by imaging the observation target, a function of setting a plurality of regions of interest in the image, a function of performing determination processing of determining, with respect to each of the plurality of regions of interest, whether or not correction of data using the region of interest is possible, and a function of correcting the data using the region of interest for which it is determined that the correction of the data is possible.

With the present invention, the data used in calculating the oxygen saturation can be more reliably and quickly corrected.

### BRIEF DESCRIPTION OF THE DRAWINGS

FIG. 1 is an explanatory view for describing a region of interest.

FIG. 2 is a schematic view of an endoscope system using a laparoscope.

FIG. 3A is an explanatory view for describing displaying on a display in a normal mode, and FIG. 3B is an explanatory view for describing displaying on an extended display in the normal mode.

FIG. 4A is an explanatory view for describing displaying on the display in an oxygen saturation mode, and FIG. 4B is an explanatory view for describing displaying on the extended display in the oxygen saturation mode.

FIG. 5 is a view showing display of a message on the extended display.

FIG. 6 is a block diagram showing functions of the endoscope system.

FIG. 7 is a graph showing a light emission spectrum of white light.

FIG. 8A is a graph showing a light emission spectrum of first illumination light, FIG. 8B is a graph showing a light emission spectrum of second illumination light, and FIG. 8C is a graph showing a light emission spectrum of green light G.

FIG. 9 is a graph showing a spectral sensitivity of an imaging sensor.

FIG. 10 is a table showing illumination and image signals to be acquired in the normal mode.

FIG. 11 is a table showing illumination and image signals to be acquired in the oxygen saturation mode or a correction mode.

FIG. 12 is an explanatory view for describing light emission control and display control in the oxygen saturation mode or the correction mode.

FIG. 13 is a graph showing different reflection spectra of hemoglobin depending on a blood concentration.

FIG. 14 is a graph showing different reflection spectra of hemoglobin depending on a concentration of a yellow coloring agent and an absorption spectrum of the yellow coloring agent.

FIG. 15 is a table showing oxygen saturation dependence, blood concentration dependence, and brightness dependence of a B1 image signal, a G2 image signal, and an R2 image signal in a case where there is no effect of the yellow coloring agent.

FIG. 16 is a graph showing contour lines representing oxygen saturation.

FIG. 17 is a table showing oxygen saturation dependence, blood concentration dependence, and brightness dependence related to a value of an X-axis indicating a signal ratio ln (R2/G2) and a value of a Y-axis showing a signal ratio ln (B1/G2).

FIG. 18 is a table showing oxygen saturation dependence, blood concentration dependence, yellow coloring agent dependence, and brightness dependence of the B1 image signal, the G2 image signal, and the R2 image signal in a case where there is an effect of the yellow coloring agent.

FIG. 19 is an explanatory view showing oxygen saturation in a case of having the yellow coloring agent and oxygen saturation in a case of not having the yellow coloring agent, in a case where an observation target has the same oxygen saturation.

FIG. 20 is a table showing oxygen saturation dependence, blood concentration dependence, yellow coloring agent dependence, and brightness dependence of the B1 image signal, a B3 image signal, G2 and G3 image signals, the R2 image signal, and a B2 image signal in a case where there is an effect of the yellow coloring agent.

FIG. 21 is a graph showing a curved surface representing oxygen saturation according to the yellow coloring agent.

FIG. 22 is an explanatory view of a case where a state of the oxygen saturation expressed by three-dimensional coordinates of X, Y, and Z is expressed by two-dimensional coordinates of X and Y FIG. 23 is a table showing oxygen saturation dependence, blood concentration dependence, yellow coloring agent dependence, and brightness dependence related to the value of the X-axis showing the signal ratio ln (R2/G2), the value of the Y-axis showing the signal ratio ln (B1/G2), and a value of a Z-axis showing a signal ratio ln (B3/G3).

FIG. 24 is a block diagram showing functions of an extended processor device.

FIG. 25 is a block diagram showing functions of an image processing unit.

FIG. 26 is an explanatory view showing a calculation method of the oxygen saturation.

FIG. 27 is an explanatory view showing a generation method of a contour line corresponding to a specific coloring agent concentration.

FIG. 28 is a block diagram showing functions of a table correction unit.

FIG. 29 is a block diagram showing functions of a determination processing unit.

FIG. 30 is an explanatory view for describing a method of performing determination processing through serial processing.

FIG. 31 is an explanatory view for describing the method of performing the determination processing through the serial processing and is an explanatory view for describing changes of regions of interest which are determined as a region of interest shown in (A) of FIG. 31, a region of interest shown in (B) of FIG. 31, a region of interest shown in (C) of FIG. 31, and a region of interest shown in (D) of FIG. 31.

FIG. 32 is a graph showing a relationship between a pixel value of the G2 image signal and a reliability degree.

FIG. 33 is a graph related to a reliability degree considering bleeding or the like.

FIG. 34 is a graph related to a reliability degree considering fat or the like.

FIG. 35 is an explanatory view for describing a method of determining a representative region.

FIG. 36 is an image view of an endoscope image in which the representative region is displayed.

FIG. 37 is an explanatory view for describing functions of the determination processing unit.

FIG. 38 is an explanatory view of a case where a region that can be corrected using a threshold value is displayed.

FIG. 39 is an explanatory view of a case where a message is displayed using the threshold value.

FIG. 40 is an explanatory view for describing a circular region of interest.

FIG. 41 is an explanatory view for describing use of regions of interest having different shapes in the endoscope image.

FIG. 42 is an explanatory view for describing an example of a method of the determination processing.

FIG. 43 is an explanatory view for describing an example of the method of the determination processing.

FIG. 44 is a graph showing correction data for correcting a calculation pixel reliability degree.

FIG. 45 is a graph showing weighting performed with respect to a region reliability degree according to an area of the region of interest.

FIG. 46 is a graph showing weighting performed with respect to a region reliability degree according to a region distance.

FIG. 47 is an image view showing a correction image group with an outline.

FIG. 48 is a flowchart for describing processing of the endoscope system.

### DESCRIPTION OF THE PREFERRED EMBODIMENTS

An example of an endoscope system according to an embodiment of the present invention will be described. First, a background leading to obtaining an example of the following embodiment will be described. Oxygen saturation imaging using an endoscope is a technique of calculating hemoglobin oxygen saturation from a small amount of spectral information of visible light. In order to calculate the oxygen saturation, for example, observation targets are imaged by a plurality of illumination light beams having different wavelength ranges, respectively. At least, light in a wavelength range in which a light absorption coefficient is different between oxygenated hemoglobin and reduced hemoglobin is used as the illumination light to image the observation target. Then, a predetermined operation value is calculated using a pixel value of the obtained image, and the operation value is set as the oxygen saturation of the observation target using an oxygen saturation calculation table representing a correlation in which the operation value is associated with the oxygen saturation. The correlation of the oxygen saturation calculation table or the like is data used in calculating the oxygen saturation.

In a case where there is a yellow coloring agent or the like in the observation target, the yellow coloring agent affects light absorption of blood hemoglobin, and thus there is a problem in which a calculation value of the oxygen saturation is shifted. A situation where the yellow coloring agent is contained varies in some cases depending on individual differences or the like of patients such as various types of parts, such as an esophagus, a stomach, and a large intestine, gender, and age.

Even in such a case, in order to more accurately calculate the oxygen saturation, the oxygen saturation calculation table can be corrected (WO2023/119856A). It is necessary to use an endoscope image obtained by appropriately imaging an observation target in correcting the oxygen saturation calculation table. In this case, appropriately imaging the endoscope image means capturing the endoscope image such that the oxygen saturation calculation table can be corrected.

On the other hand, a technique of making a notification in a case where the correction of the oxygen saturation calculation table has failed due to halation, movement, or the like is known (JP2017-080246A). In addition, in particular, in a case where there is a yellow coloring agent or the like in an observation target, the yellow coloring agent affects light absorption of blood hemoglobin, and thus there is a problem in which a calculation value of oxygen saturation is shifted. Meanwhile, a technique of displaying the above information on a display (WO2023/119856A) is known.

However, in the technique, an endoscope image with which the correction of the oxygen saturation calculation table does not fail is found by trial and error in some cases. It takes a long time to correct the oxygen saturation calculation table, or more effort is required for an endoscope user (hereinafter, referred to as a user) in some cases.

In the endoscope system, an image processing apparatus, an operation method of an endoscope system, and an endoscope system program according to the embodiment of the present invention, a plurality of regions of interest are set in an endoscope image, and it is determined whether or not the region is a region with which data used in calculating oxygen saturation can be corrected for each region of interest. Therefore, even in a case where a plurality of parts are captured in the endoscope image or in a case where halation occurs, bleeding occurs in an observation target, or the like, whether the region is a region with which the oxygen saturation calculation table can be corrected for each region of interest included in the endoscope image is determined, and the oxygen saturation calculation table is corrected using the region with which the oxygen saturation calculation table can be corrected. Thus, the oxygen saturation calculation table can be more reliably corrected. In addition, since the processing is automatically performed, it is not necessary for the user to perform excessive trial and error, and the correction of the oxygen saturation calculation table can be performed after identifying a region with which the correction succeeds more reliably and quickly from the endoscope image. Therefore, with the present invention, the oxygen saturation calculation table, which is data used in calculating the oxygen saturation, can be more reliably and quickly corrected.

The endoscope system according to the embodiment of the present invention is an endoscope system that calculates oxygen saturation of an observation target using data used in calculating the oxygen saturation and comprises a processor device. Specifically, the processor device is a computer comprising a processor, a memory, and the like. An extended processor device may be connected to the processor device, or the extended processor device may be configured to realize a function of the processor device. In addition, the processor device may be configured to realize the function of the extended processor device. The processor device or the extended processor device comprises an image processing unit, and the image processing unit comprises an image acquisition unit, a region setting unit, a determination processing unit, and a correction unit.

The image acquisition unit acquires an image obtained by imaging an observation target. The observation target is imaged by an endoscope such as a flexible endoscope and a rigid endoscope. The observation target is, for example, an organ such as a large intestine and a small intestine imaged by a laparoscope which is the rigid endoscope, and specifically, a serous membrane or the like of the organ. The image acquisition unit acquires, for example, an endoscope image captured by the laparoscope. The region setting unit sets a plurality of regions of interest in the image acquired by the image acquisition unit.

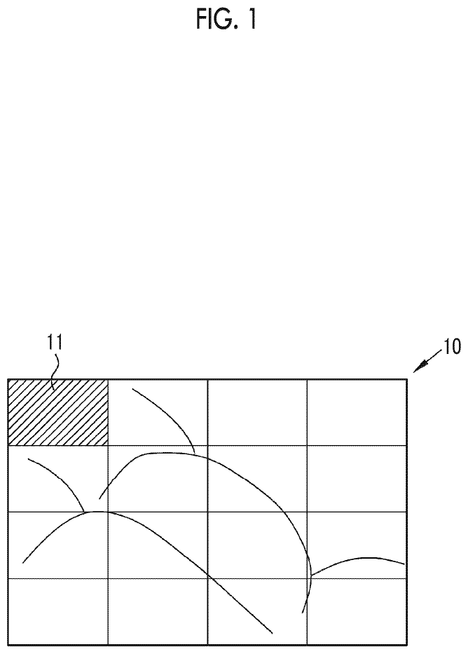

As shown in FIG. 1, the region setting unit sets a plurality of regions of interest 11 in an endoscope image 10. In FIG. 1, 16 regions of interest 11 are set in the endoscope image 10. In FIG. 1, a region indicated by diagonal lines is one region of interest 11. In addition, in FIG. 1, the reference numeral is given only to some of the regions of interest 11 in order to avoid complication. The shapes, sizes, and the like of the regions of interest 11 may be set in any manner as long as the plurality of regions of interest 11 can be set in the endoscope image.

The determination processing unit performs determination processing of determining whether or not data used in calculating oxygen saturation can be corrected with respect to each of the plurality of regions of interest 11 obtained by dividing the endoscope image 10. Examples of the data used in calculating oxygen saturation include the oxygen saturation calculation table. As a method of correcting the oxygen saturation calculation table, a method of using image information of the endoscope image 10 is used. Therefore, in the case of FIG. 1, the determination processing unit performs the determination processing with respect to each of 16 regions of interest 11. The determination processing may be performed through parallel processing with respect to all of the plurality of regions of interest 11 or through serial processing in which one or a specific number of the plurality of regions of interest 11 are processed in turn.

The region of interest 11 with which the oxygen saturation calculation table can be corrected means a region of which oxygen saturation can be appropriately obtained by correcting the oxygen saturation calculation table using the region of interest 11. In a case where the oxygen saturation is calculated after the oxygen saturation calculation table is corrected using the region of interest 11 with which the oxygen saturation calculation table can be corrected, the obtained oxygen saturation is more accurate than in a case where the oxygen saturation is calculated without the correction. Alternatively, even in a case where the oxygen saturation cannot be calculated in a case where the oxygen saturation calculation table is not corrected, the oxygen saturation can be obtained using the corrected oxygen saturation calculation table.

On the other hand, the region of interest 11 with which the oxygen saturation calculation table cannot be corrected means a region of which the oxygen saturation obtained by correcting the oxygen saturation calculation table based on the region of interest 11 is inappropriate. In a case where the oxygen saturation is calculated after the oxygen saturation calculation table is corrected using the region of interest 11 with which the oxygen saturation calculation table cannot be corrected, the obtained oxygen saturation is more inaccurate than in a case where the oxygen saturation is calculated without the correction. In addition, correction itself of the oxygen saturation calculation table cannot be corrected in some cases using the region of interest 11 with which the oxygen saturation calculation table cannot be corrected.

In the region of interest 11, as the determination processing of determining whether or not the oxygen saturation calculation table can be corrected, processing of appropriately setting an indicator related to obtaining the oxygen saturation in a case where the oxygen saturation calculation table is corrected using the region of interest 11 and performing the determination based on the indicator can be performed. As the indicator, a reliability degree can be set and used. The reliability degree is set using the image information of the region of interest 11.

The correction unit corrects the oxygen saturation calculation table using the region of interest 11 for which it is determined that the correction of the oxygen saturation calculation table is possible. In a case where there is no region of interest 11 with which the oxygen saturation calculation table can be corrected in the determination result from the determination processing, the oxygen saturation calculation table is not corrected. In a case where there are a plurality of regions of interest 11 with which the oxygen saturation calculation table can be corrected, the oxygen saturation calculation table is corrected through a method set in advance. Examples of the method set in advance include a method of using the region of interest 11 that is most appropriate to be used in correcting the oxygen saturation calculation table, which is selected using the reliability degree, that is, a method of displaying the regions of interest 11 for which it is determined that the correction of the oxygen saturation calculation table is possible on the display, designating one region of interest 11 to be used in correcting the oxygen saturation calculation table from the displayed regions of interest 11 by the user, and correcting the oxygen saturation calculation table using all of the regions of interest 11.

As described above, since the endoscope system performs the determination processing of determining whether or not data used in calculating oxygen saturation can be corrected with respect to each of the plurality of regions of interest 11 in an endoscope image in the correction of the data used in calculating oxygen saturation, even in a case where a region that is a problem related to the correction of the oxygen saturation calculation table is included in the endoscope image, the oxygen saturation calculation table is corrected after identifying a region that is not a problem, a region that is most suitable for the correction, and the like. Therefore, the oxygen saturation calculation table can be more reliably corrected without excessive trial and error. In addition, processing in the image processing unit is, for example, automatically performed for each frame image, and the user does not need to perform an additional operation. Thus, the oxygen saturation calculation table can be quickly corrected. Therefore, the endoscope system having the above configuration can more reliably and quickly correct the data used in calculating oxygen saturation.

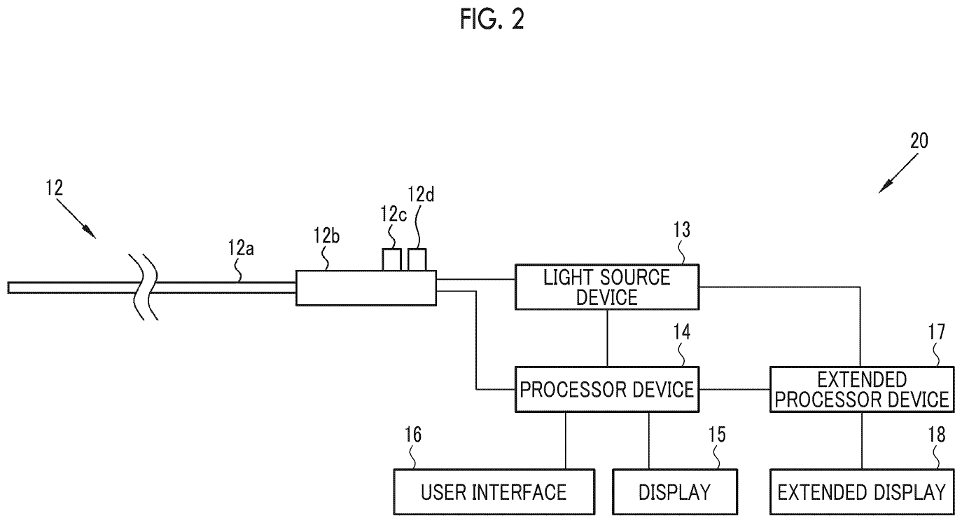

Hereinafter, the embodiment will be described with reference to the drawings. As shown in FIG. 2, an endoscope system 20 comprises an endoscope 12, a light source device 13, a processor device 14, a display 15, a user interface 16, an extended processor device 17, and an extended display 18. The endoscope 12 is optically or electrically connected to the light source device 13 and electrically connected to the processor device 14. The extended processor device 17 is electrically connected to the light source device 13 and the processor device 14. The connection of each of these is not limited to a wired connection and may be a wireless connection. In addition, the connection may be made via a network. In addition, in the present specification, the display includes the display 15 and the extended display 18.

The endoscope 12 is a rigid endoscope that is inserted into a body cavity of a subject in order to perform a surgical treatment and that images an organ in the body cavity from a serosal side and is a laparoscope. The endoscope 12 may be a flexible endoscope inserted from a nose, a mouth, or an anus of the subject. In addition, in the present specification, the subject means a target into which the endoscope 12 is inserted. The subject includes an observation target.

The endoscope 12 comprises an insertion part 12a inserted into an abdominal cavity of a subject and an operating part 12b provided at a proximal end portion of the insertion part 12a. An illumination optical system and an imaging optical system are built in a portion in the vicinity of a distal end (hereinafter, referred to as a distal end portion) of the insertion part 12a. An image signal is generated by the systems. The generated image signal is output to the processor device 14. The operating part 12b is provided with a mode switching switch 12c and a zoom operation switch 12d for a zoom operation or the like. The mode switching switch 12c is used in a switching operation of the observation modes.

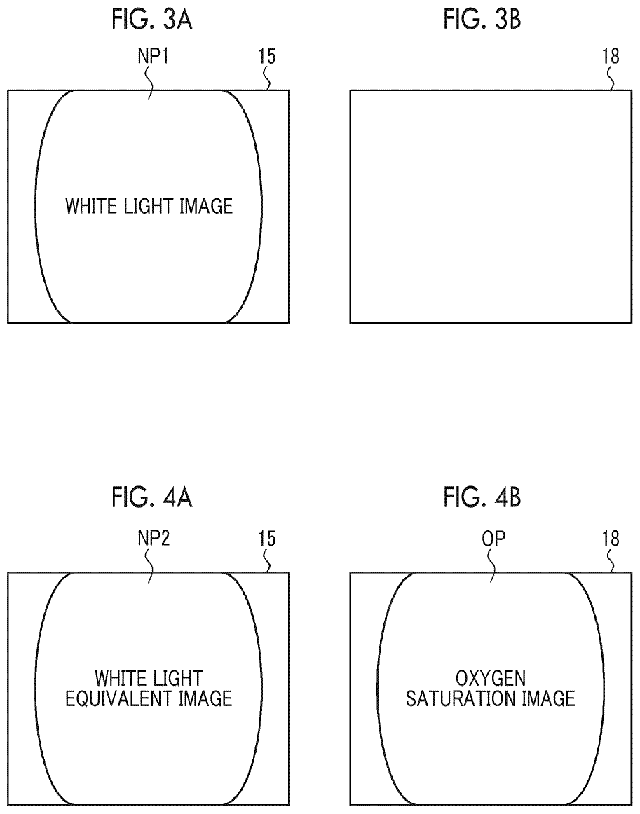

The endoscope system 20 has three modes including a normal mode, an oxygen saturation mode, and a tissue color correction mode (hereinafter, referred to as a correction mode). As shown in FIGS. 3A and 3B, in the normal mode, while a natural color white light image NP1 obtained by imaging an observation target using illumination light and white light is displayed on the display 15, nothing is displayed on the extended display 18.

As shown in FIGS. 4A and 4B, in the oxygen saturation mode, oxygen saturation of an observation target is calculated based on an endoscope image obtained by imaging the observation target, and an oxygen saturation image OP obtained by visualizing the calculated oxygen saturation is displayed on the extended display 18. In addition, in the oxygen saturation mode, a white light equivalent image NP2 in which a short wavelength component is less than that in the white light image is displayed on the display 15 due to a difference between illumination light in the normal mode and illumination light in the oxygen saturation mode.

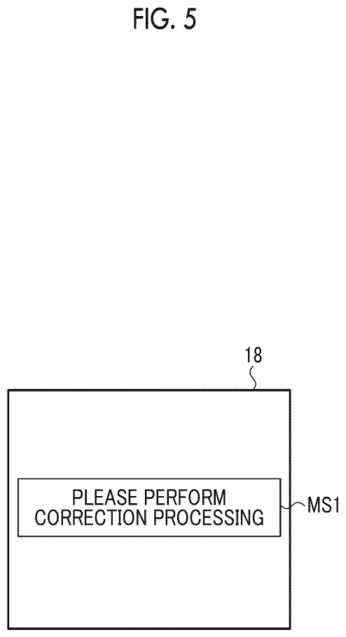

In the correction mode, the white light equivalent image NP2 is displayed on the extended display 18. In the correction mode, correction processing of the oxygen saturation calculation table is performed using the region of interest 11 for which it is determined that the correction of the oxygen saturation calculation table is possible. In a case of switching from the normal mode to the oxygen saturation mode, the mode may be switched to the oxygen saturation mode after the mode is automatically switched to the correction mode temporarily and the correction processing is completed in the correction mode. In addition, in a case where the mode is switched from the normal mode to the oxygen saturation mode, the user may be notified of whether or not to perform the correction processing in the correction mode, and the user may determine whether or not to perform the correction mode. In addition, as shown in FIG. 5, in a case of switching to the oxygen saturation mode, a message MS1 of “Please perform correction processing” may be displayed on the extended display 18. In response to the message MS1, the user can switch to the correction mode without forgetting the correction processing in a case where the correction processing is necessary before the oxygen saturation mode is started. After the correction processing is completed, an oxygen saturation image is displayed on the extended display 18.

The processor device 14 controls the entire endoscope system 20. In addition, an endoscope image is generated by performing image processing or the like with respect to an image signal transmitted from the endoscope 12. The display 15 displays the endoscope image or the like generated by the processor device 14.

The user interface 16 is an interface for performing an input operation, such as device setting, with respect to the processor device 14 and is a keyboard, a mouse, a microphone, a tablet, a foot switch, or the like. In a case where the display 15 or the extended display 18 includes a touch panel, the display 15 or the extended display 18 may also serve as the user interface 16.

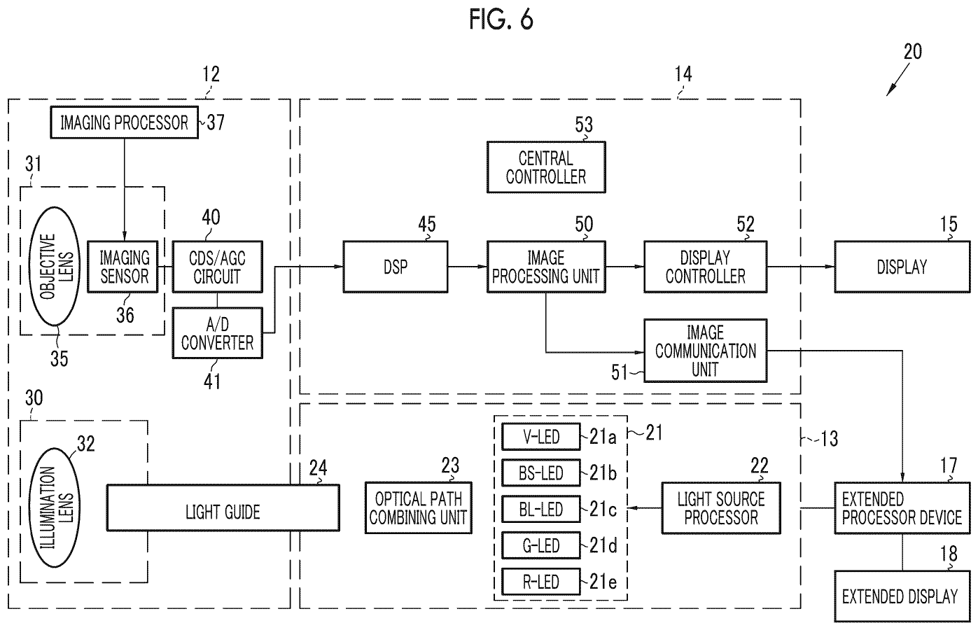

As shown in FIG. 6, the light source device 13 comprises a light source unit 21 and a light source processor 22 that controls the light source unit 21. The light source unit 21 includes, for example, a plurality of semiconductor light sources, turns on or off each of the semiconductor light sources, and emits illumination light, which illuminates an observation target, by controlling the amount of light emitted from each semiconductor light source, in a case of turning on.

In the present embodiment, the light source unit 21 includes five color LEDs including a violet light emitting diode (V-LED) 21a, a blue short-wavelength light emitting diode (BS-LED) 21b, a blue long-wavelength light emitting diode (BL-LED) 21c, a green light emitting diode (G-LED) 21d, and a red light emitting diode (R-LED) 21e.

Instead of the LEDs, a combination of a laser diode (LD), a phosphor, and a band limitation filter or a combination of a lamp, such as a xenon lamp, and a band limitation filter can be used as the light source unit 21.

The V-LED 21a emits violet light V (hereinafter, referred to as V light) of 410 nm±10 nm. The BS-LED 21b emits second blue light BS of 450 nm±10 nm. The BL-LED 21c emits first blue light BL of 470 nm±10 nm. The G-LED 21d emits green light G in a green band. It is preferable that a central wavelength of the green light G is 540 nm. The R-LED 21e emits red light R in a red band. It is preferable that a central wavelength of the red light R is 620 nm. A central wavelength and a peak wavelength of each of the LEDs 21a to 21e may be the same or may be different from each other.

Both the BL-LED 21c and the BS-LED 21b are blue light sources that emit blue light. However, the central wavelengths and the wavelength ranges of the blue light emitted from the BL-LED 21c (hereinafter, referred to as BL light) and the blue light emitted from the BS-LED 21b (hereinafter, referred to as BS light) are different from each other as described above. The central wavelength and the wavelength range of the BL light are a central wavelength and a wavelength range in which a difference in light absorption coefficients between oxidized hemoglobin and reduced hemoglobin is mostly maximized in a blue wavelength range.

The G-LED 21d is a green light source that emits wideband green light having a central wavelength of 540 nm (hereinafter, referred to as G light). The R-LED 21e is a red light source that emits wideband red light (hereinafter, referred to as R light).

The light source processor 22 independently controls turning on or off of each of the LEDs 21a to 21e, the amount of light emitted in a case of turning on, and the like by independently inputting a control signal to each of the LEDs 21a to 21e. The control of the light source processor 22, such as turning on or off, is different depending on each mode, and details thereof will be described later.

Light emitted from each of the LEDs 21a to 21e is incident to a light guide 24 via an optical path combining unit 23 composed of a mirror, a lens, and the like. The light guide 24 is built in the endoscope 12 and a universal cord (a cord connecting the endoscope 12, the light source device 13, and the processor device 14). The light guide 24 propagates light from the optical path combining unit 23 to a distal end portion of the endoscope 12.

The endoscope 12 is provided with an illumination optical system 30 and an imaging optical system 31. The illumination optical system 30 has an illumination lens 32, and an observation target is irradiated with illumination light, which is propagated by the light guide 24, via the illumination lens 32. The imaging optical system 31 has an objective lens 35 and an imaging sensor 36. Light from the observation target, which is irradiated with the illumination light, is incident to the imaging sensor 36 via the objective lens 35. Accordingly, an image of the observation target is formed on the imaging sensor 36.

The imaging sensor 36 is a color imaging sensor that images an observation target which is being illuminated with illumination light. Each pixel of the imaging sensor 36 is provided with any one of a blue pixel (B pixel) having a blue (B) color filter, a green pixel (G pixel) having a green (G) color filter, or a red pixel (R pixel) having a red (R) color filter. For this reason, in a case where the imaging sensor 36 images the observation target, three types of images, that is, a B image, a G image, and an R image, are obtained. The imaging sensor 36 is preferably a color imaging sensor of a Bayer array in which a ratio of the number of pixels of the B pixels, the G pixels, and the R pixels is 1:2:1.

A charge-coupled device (CCD) imaging sensor or a complementary metal-oxide-semiconductor (CMOS) imaging sensor can be used as the imaging sensor 36. In addition, a complementary color imaging sensor comprising complementary color filters corresponding to cyan (C), magenta (M), yellow (Y), and green (G) may be used instead of the primary color imaging sensor 36. In a case where the complementary color imaging sensor is used, image signals corresponding to four colors of C, M, Y, and G are output. Therefore, by converting the image signals corresponding to the four colors of C, M, Y, and G into image signals corresponding to three colors of R, G, and B through complementary color-primary color conversion, the image signals corresponding to respective colors of R, G, and B which are the same as those of the imaging sensor 36 can be obtained.

The imaging sensor 36 is drive-controlled by an imaging processor 37. The control of each mode in the imaging processor 37 will be described later. A correlated double sampling/automatic gain control (CDS/AGC) circuit 40 performs correlated double sampling (CDS) or automatic gain control (AGC) on an analog image signal obtained from the imaging sensor 36. An image signal that has passed via the CDS/AGC circuit 40 is converted into a digital image signal by an analog/digital (A/D) converter 41. The digital image signal, which has been subjected to A/D conversion, is input to the processor device 14.

The processor device 14 comprises a digital signal processor (DSP) 45, an image processing unit 50, an image communication unit 51, a display controller 52, and a central controller 53. In the processor device 14, a program related to various types of processing is incorporated in a program memory (not shown). As the central controller 53 composed of a processor executes the program in the program memory, functions of the DSP 45, the image processing unit 50, the image communication unit 51, the display controller 52, and the central controller 53 are realized.

The DSP 45 performs various types of signal processing, such as defect correction processing, offset processing, gain correction processing, demosaicing processing, linear matrix processing, white balance processing, gamma conversion processing, YC conversion processing, and noise reduction processing, with respect to an image signal received from the endoscope 12. In the defect correction processing, a signal of a defective pixel of the imaging sensor 36 is corrected. In the offset processing, a dark current component is removed from the image signal on which the defect correction processing is performed, and an accurate zero level is set. In the gain correction processing, a signal level of each image signal is adjusted by multiplying the image signal of each color after the offset processing by a specific gain. The demosaicing processing and the linear matrix processing for improving color reproducibility are performed on the image signal of each color after the gain correction processing.

After the linear matrix processing, the white balance processing is performed, and then brightness and chroma saturation of each image signal are adjusted through the gamma conversion processing. After that, the YC conversion processing is performed, and a brightness signal Y, a color difference signal Cb, and a color difference signal Rc are output to the DSP 45. The DSP 45 performs the noise reduction processing through, for example, a moving average method or a median filter method.

The image processing unit 50 performs various types of image processing with respect to an image signal from the DSP 45. The image processing includes 3×3 matrix processing, gradation transformation processing, color conversion processing such as three-dimensional oxygen saturation calculation table processing, color enhancement processing, and structure enhancement processing such as spatial frequency enhancement. The image processing unit 50 performs image processing according to the mode. In the case of the normal mode, the image processing unit 50 generates the white light image NP1 by performing normal mode image processing. In the case of the oxygen saturation mode, the image processing unit 50 generates the white light equivalent image NP2 and transmits an image signal from the DSP 45 to the extended processor device 17 via the image communication unit 51. In the extended processor device 17, the oxygen saturation image OP is generated based on the transmitted image signal of the endoscope image.

The display controller 52 performs display control for displaying image signals, such as the white light image NP1 and the oxygen saturation image OP, other information, and the like from the image processing unit 50, on the display 15.

The extended processor device 17 receives an image signal from the processor device 14 and performs various types of image processing. The extended processor device 17 functions as the image processing apparatus. The image processing in the extended processor device 17 is performed in the correction mode and the oxygen saturation mode and is processing for generating the oxygen saturation image.

The extended processor device 17 calculates oxygen saturation in the oxygen saturation mode and generates the oxygen saturation image OP in which the calculated oxygen saturation is visualized. The generated oxygen saturation image OP is displayed on the extended display 18. In addition, the extended processor device 17 performs correction processing related to the calculation of the oxygen saturation in the case of the correction mode. More specifically, in the correction processing, a specific coloring agent concentration is calculated in response to a user operation, and the oxygen saturation calculation table is corrected based on the calculated specific coloring agent concentration. Examples of a specific coloring agent include a yellow coloring agent. Therefore, the specific coloring agent concentration is a concentration of the yellow coloring agent in the image information included in the endoscope image 10. Details of the oxygen saturation mode and the correction mode performed by the extended processor device 17 will be described later.

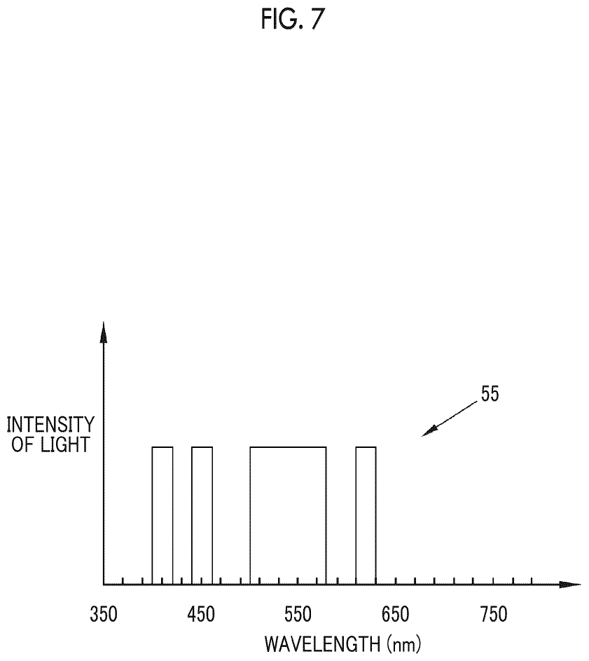

The control of turning on or off in each mode will be described. In the normal mode, as the V-LED 20a, the BS-LED 20b, the G-LED 20d, and the R-LED 20e are simultaneously turned on, white light 55, including the violet light V having a central wavelength of 410 nm, the second blue light BS having a central wavelength of 450 nm, the wideband green light G in the green light band, and the red light R having a central wavelength of 620 nm, is emitted as shown in FIG. 7. The graph shown in the drawing schematically shows the intensity of light in each wavelength range.

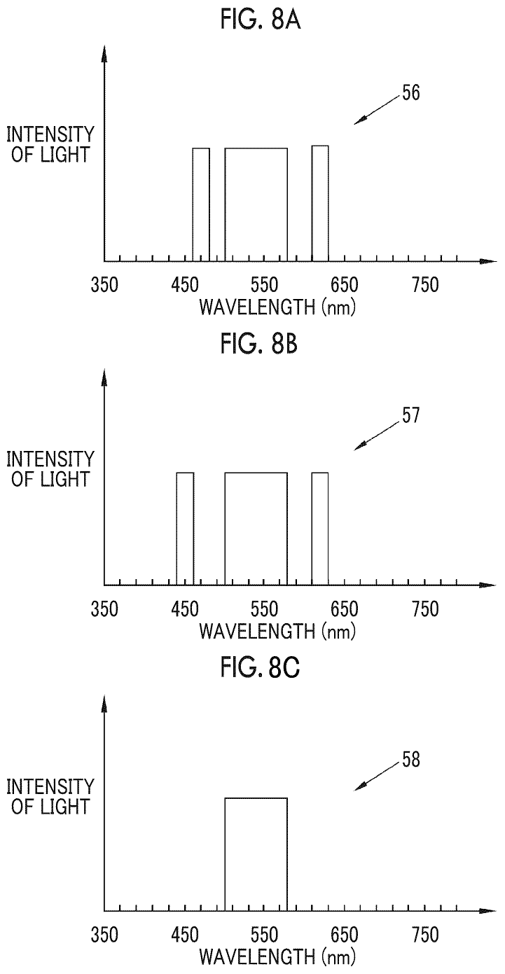

In the oxygen saturation mode and the correction mode, light emission of three frames in which light emission patterns are different from each other is repeatedly performed. In the first frame, as shown in FIG. 8A, as the BL-LED 20c, the G-LED 20d, and the R-LED 20e are simultaneously turned on, wideband first illumination light 56, including the first blue light BL having a central wavelength of 470 nm, the wideband green light G in the green band, and the red light R having a central wavelength of 620 nm, is emitted. In the second frame, as shown in FIG. 8B, as the BS-LED 20b, the G-LED 20d, and the R-LED 20e are simultaneously turned on, second illumination light 57, including the second blue light BS having a central wavelength of 450 nm, the wideband green light G in the green band, and the red light R having a central wavelength of 620 nm, is emitted. In the third frame, as shown in FIG. 8C, as the G-LED 20d is turned on, the wideband green light G in the green band is emitted as third illumination light 58. In the oxygen saturation mode, since frames necessary to obtain image signals required in calculating the oxygen saturation are the first frame and the second frame, light may be emitted in only the first frame and the second frame.

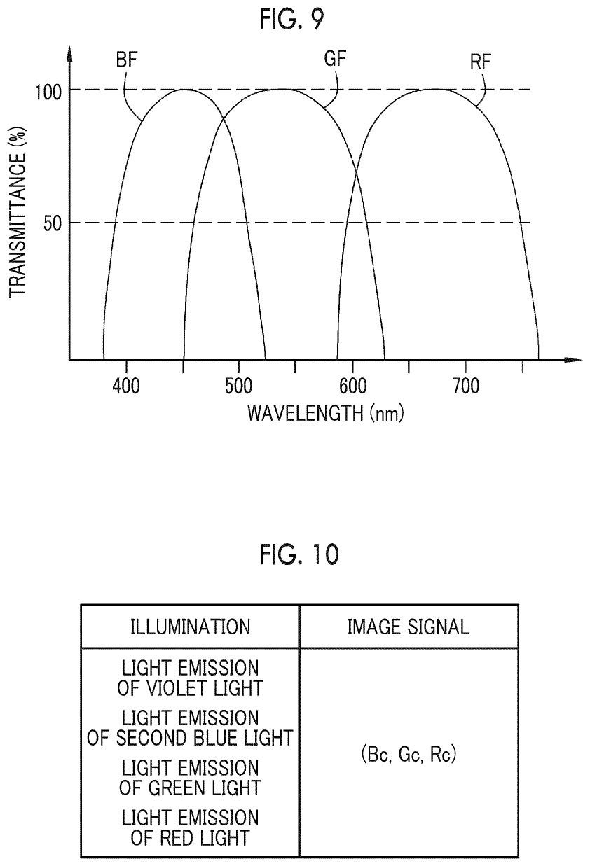

As shown in FIG. 9, a B color filter BF provided in the B pixel of the imaging sensor 36 mainly transmits light in a blue band, specifically, light in a wavelength range of 380 to 560 nm (blue transmission range). A peak wavelength at which a transmittance is at its maximum is in the vicinity of 460 to 470 nm. A G color filter GF provided in the G pixel of the imaging sensor 36 mainly transmits light in the green band, specifically, light having a wavelength range of 450 to 630 nm (green transmission range). An R color filter RF provided in the R pixel of the imaging sensor 36 mainly transmits light in the red band, specifically, light of 580 to 760 nm (red transmission range).

As shown in FIG. 10, in the normal mode, the imaging processor 37 controls the imaging sensor 36 such that an observation target illuminated with the white light 55, including the violet light V, the second blue light BS, the green light G, and the red light R, is imaged for each frame. Accordingly, a Bc image signal is output from the B pixel of the imaging sensor 36, a Gc image signal is output from the G pixel thereof, and an Rc image signal is output from the R pixel thereof.

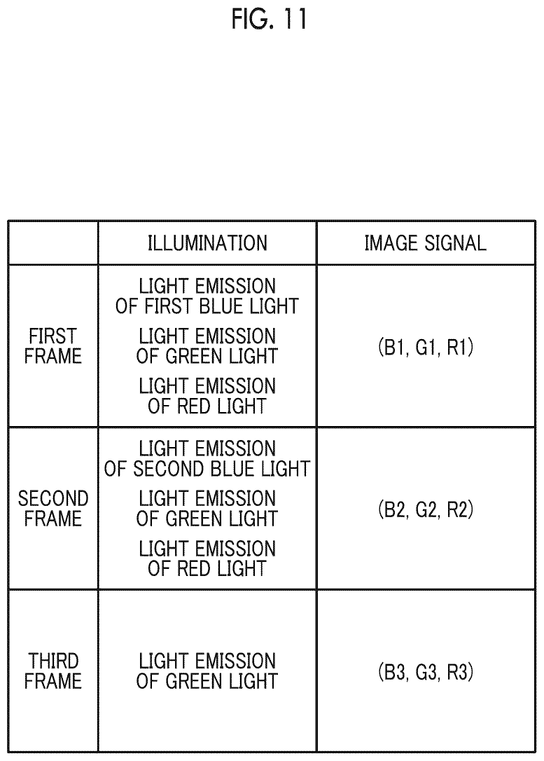

As shown in FIG. 11, in the oxygen saturation mode, in a case where an observation target is illuminated with the first illumination light 56, including the first blue light BL, the green light G, and the red light R, in the first frame, a B1 image signal is output from the B pixel, a G1 image signal is output from the G pixel, and an R1 image signal is output from the R pixel of the imaging sensor 36 as a first illumination light image by the imaging processor 37. In a case where the observation target is illuminated with the second illumination light 57, including the second blue light BS, the green light G, and the red light R, in the second frame, a B2 image signal is output from the B pixel, a G2 image signal is output from the G pixel, and an R2 image signal is output from the R pixel of the imaging sensor 36 as a second illumination light image by the imaging processor 37.

In a case where an observation target is illuminated with the third illumination light 58 which is the green light G in the third frame, a B3 image signal is output from the B pixel of the imaging sensor 36, a G3 image signal is output from the G pixel, and an R3 image signal is output from the R pixel as a third illumination light image by the imaging processor 37.

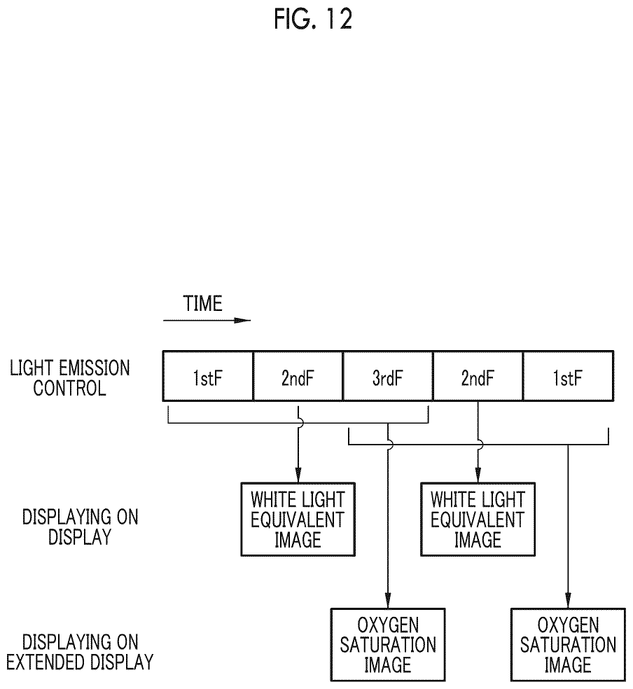

In the oxygen saturation mode, as shown in FIG. 12, after the first illumination light 56 is emitted in the first frame (1st F), the second illumination light 57 is emitted in the second frame (2nd F), and the third illumination light 58 is emitted in the third frame (3rd F), the second illumination light 57 in the second frame is emitted, and the first illumination light 56 in the first frame is emitted. The white light equivalent image NP2 obtained based on the light emission of the second illumination light 57 in the second frame is displayed on the display 15. In addition, the oxygen saturation image OP obtained based on the light emission of the first to third illumination lights in the first to third frames is displayed on the extended display 18.

In the oxygen saturation mode, among image signals of the above three frames, the B1 image signal included in the first illumination light image and the G2 image signal and the R2 image signal which are included in the second illumination light image are used. In addition, in the correction mode, in order to measure the concentration of a specific coloring agent (a yellow coloring agent and the like) that affects the calculation accuracy of the oxygen saturation, the B3 image signal and the G3 image signal included in the third illumination light image are used in addition to the B1 image signal, the G2 image signal, and the R2 image signal.

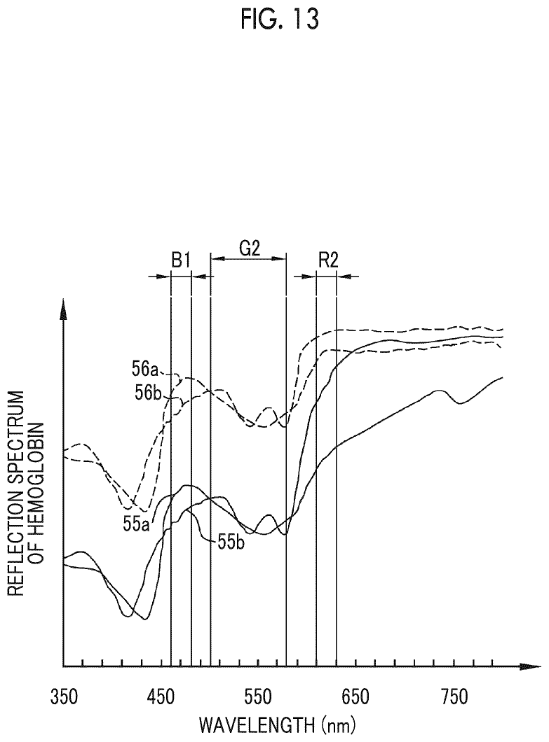

The B1 image signal includes at least image information related to the first blue light BL among light transmitted through the B color filter BF in the first illumination light 56. The B1 image signal (oxygen saturation image signal) includes image information of a wavelength range B1 in which a reflection spectrum changes due to a change in the oxygen saturation of blood hemoglobin as the image information related to the first blue light BL. As shown in FIG. 13, for example, the wavelength range B1 is preferably a wavelength range of 460 nm to 480 nm including 470 nm at which a difference between the reflection spectrum of oxygenated hemoglobin indicated by curves 55b and 56b and the reflection spectrum of reduced hemoglobin indicated by the curves 55a and 56a is maximized.

In FIG. 13, the curve 55a represents a reflection spectrum of reduced hemoglobin in a case where a blood concentration is high, and the curve 55b represents a reflection spectrum of oxygenated hemoglobin in a case where the blood concentration is high. On the other hand, the curve 56a represents a reflection spectrum of reduced hemoglobin in a case where the blood concentration is low, and the curve 56b represents a reflection spectrum of oxygenated hemoglobin in a case where the blood concentration is low.

The G2 image signal includes at least image information of a wavelength range G2 related to the green light G among light transmitted through the G color filter GF in the first illumination light 56. For example, as shown in FIG. 13, the wavelength range G2 is preferably a wavelength range of 500 nm to 580 nm. The R2 image signal includes at least image information of a wavelength range R2 related to the red light R among light transmitted through the R color filter RF in the first illumination light 56. For example, as shown in FIG. 13, the wavelength range R2 is preferably a wavelength range of 610 nm to 630 nm.

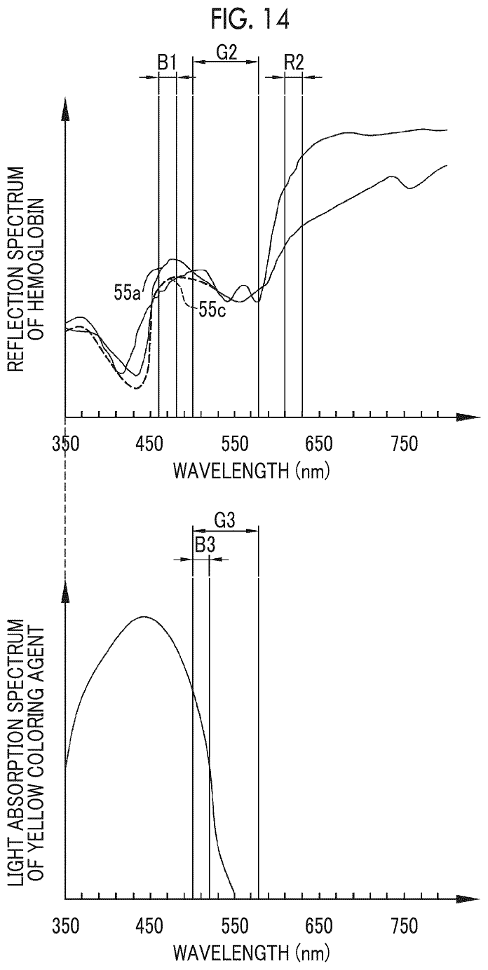

In addition, as shown in FIG. 14, image information of the wavelength range B1 includes image information related to the first blue light BL, and image information of a wavelength range B3 includes image information related to the green light G. The image information related to the first blue light BL and the green light G is image information in which a light absorption spectrum of a specific coloring agent, such as a yellow coloring agent, changes due to a change in the concentration of the specific coloring agent. As the light absorption spectrum of the specific coloring agent changes, the reflection spectrum of hemoglobin also changes. The curve 55a represents a reflection spectrum of reduced hemoglobin in a case where there is no effect of the yellow coloring agent, and a curve 55c represents a reflection spectrum of reduced hemoglobin in a case where there is an effect of the yellow coloring agent. As shown by the curves 55a and 55c, the reflection spectrum of the reduced hemoglobin changes depending on the presence or absence of the yellow coloring agent (the same applies to the reflection spectrum of oxygenated hemoglobin). Therefore, the wavelength range B1 and the wavelength range B3 receive the effect of the specific coloring agent such as the yellow coloring agent, and the reflection spectrum changes due to the oxygen saturation of blood hemoglobin.

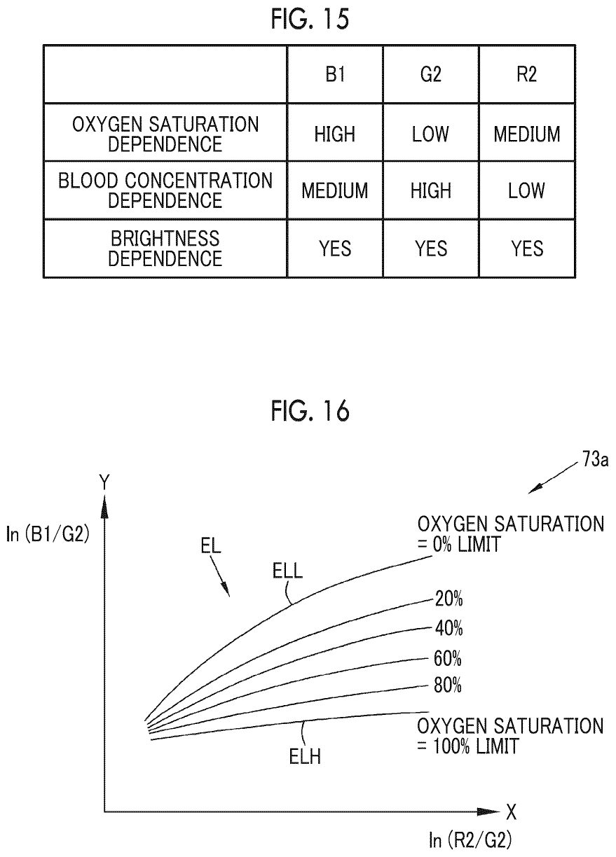

In an ideal case where there is no effect of a specific coloring agent such as a yellow coloring agent in an observation target observed using the endoscope 12, as shown in FIG. 15, each of the B1 image signal (denoted as “B1”), the G2 image signal (denoted as “G2”), and the R2 image signal (denoted as “R2”) is affected by oxygen saturation dependence, blood concentration dependence, or brightness dependence. As described above, since the B1 image signal includes the wavelength range B1 in which a difference between the reflection spectrum of the oxygenated hemoglobin and the reflection spectrum of the reduced hemoglobin is maximized, the oxygen saturation dependence that changes depending on the oxygen saturation is the degree of “high”. In addition, as shown by the curves 55a and 55b and the curves 56a and 56b, the blood concentration dependence of the B1 image signal, which changes depending on the blood concentration, is the degree of “medium”. In addition, the B1 image signal has a brightness dependence that changes depending on the brightness of the observation target. The terms “high”, “medium”, and “low” are used as degrees of dependence, but the term “high” represents that the dependence is high compared to the other image signals, the term “medium” represents that the dependence is medium compared to the other image signals, and the term “low” represents that the dependence is low compared to the other image signals.

Since a magnitude relationship between the reflection spectrum of the oxygenated hemoglobin and the reflection spectrum of the reduced hemoglobin is reversed in a wideband wavelength range, the oxygen saturation dependence of the G2 image signal is “low”. In addition, as shown by the curves 55a and 55b and the curves 56a and 56b, the blood concentration dependence of the G2 image signal is the degree of “high”. In addition, the brightness dependence of the G2 image signal is “yes” as in the B1 image signal.

The R2 image signal does not change depending on the oxygen saturation as much as the B1 image signal, but the oxygen saturation dependence is the degree of “medium”. In addition, as shown by the curves 55a and 55b and the curves 56a and 56b, the blood concentration dependence of the R2 image signal is the degree of “low”. In addition, the brightness dependence of the G2 image signal is “yes” as in the B1 image signal.

As described above, since all of the B1 image signal, the G2 image signal, and the R2 image signal have brightness dependence, an oxygen saturation calculation table 73a (see FIG. 16), which is an oxygen saturation calculation table of data for calculating oxygen saturation using a signal ratio ln (B1/G2) obtained by normalizing the B1 image signal with the G2 image signal and a signal ratio ln (R2/G2) obtained by normalizing the R2 image signal with the G2 image signal, is created using the G2 image signal as a normalization signal. The symbol “ln” in the signal ratio ln (B1/G2) is a natural logarithm (the same applies to the signal ratio ln (R2/G2)).

In a case where a relationship between the signal ratio ln (B1/G2) and the signal ratio ln (R2/G2) and the oxygen saturation is represented by two-dimensional coordinates in which the signal ratio ln (B1/G2) is represented by an X-axis and the signal ratio ln (R2/G2) is represented by a Y-axis, the oxygen saturation is represented by a contour line EL along a Y-axis direction as shown in FIG. 16. A contour line ELH represents that the oxygen saturation is “100%”, and a contour line ELL represents that the oxygen saturation is “0%”. The contour lines are distributed from the contour line ELH toward the contour line ELL such that the oxygen saturation gradually decreases (in FIG. 16, the contour lines of “80%”, “60%”, “40%”, and “20%” are distributed).

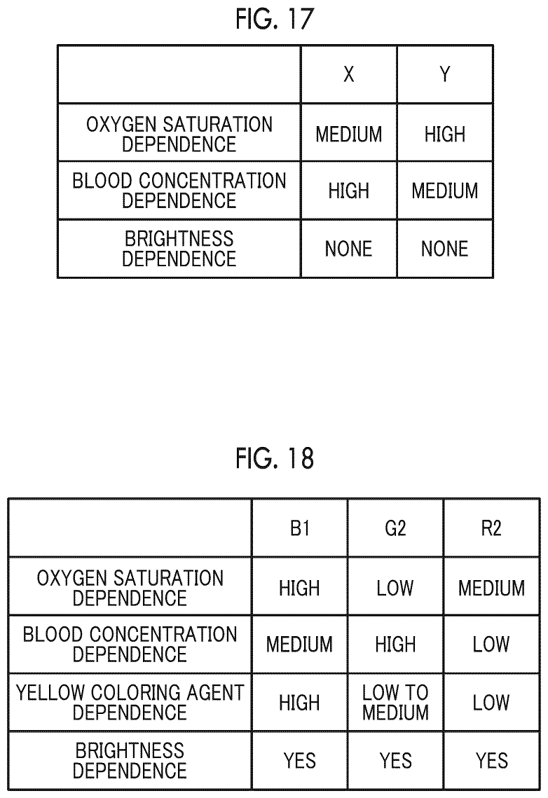

Each of the value of the X-axis (signal ratio ln (R2/G2)) and the value of the Y-axis (signal ratio ln (B1/G2)) is affected by oxygen saturation dependence and blood concentration dependence. However, regarding the brightness dependence, as shown in FIG. 17, since each of the value of the X-axis and the value of the Y-axis is normalized by the G2 image signal, “none” is set, which means not being affected. Regarding the value of the X-axis (denoted as “X”), oxygen saturation dependence is the degree of “medium”, and blood concentration dependence is the degree of “high”. On the other hand, regarding the value of the Y axis (denoted as “Y”), oxygen saturation dependence is the degree of “high”, and blood concentration dependence is the degree of “medium”.

On the other hand, in a realistic case where a specific coloring agent such as a yellow coloring agent affects an observation target observed using the endoscope 12, as shown in FIG. 18, each of the B1 image signal (denoted as “B1”), the G2 image signal (denoted as “G2”), and the R2 image signal (denoted as “R2”) is affected by oxygen saturation dependence, blood concentration dependence, yellow coloring agent dependence, or brightness dependence. Since the B1 image signal includes image information in which the light absorption spectrum of the specific coloring agent changes due to a change in the concentration of the specific coloring agent such as the yellow coloring agent, the yellow coloring agent dependence that changes depending on the yellow coloring agent is the degree of “high”. On the other hand, since the G2 image signal changes less due to the yellow coloring agent compared to the B1 image signal, the yellow coloring agent dependence is the degree of “low to medium”. Since the R1 image signal changes less due to the yellow coloring agent, the yellow coloring agent dependence is the degree of “low”.

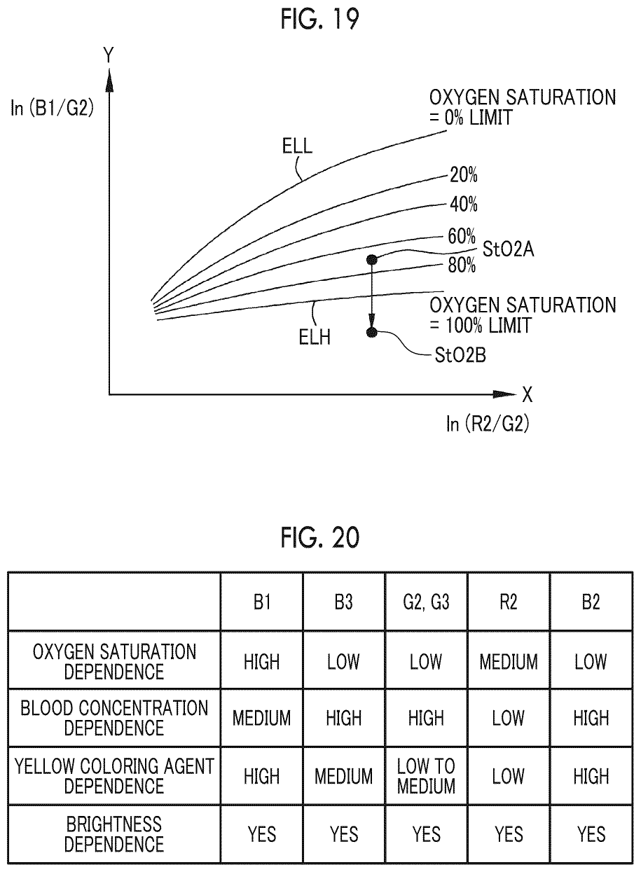

In addition, even in a case of representing in two-dimensional coordinates in which the signal ratio ln (R2/G2) is represented by the X-axis and the signal ratio ln (B1/G2) is represented by the Y-axis and in a case where there is the same oxygen saturation in the observation target, as shown in FIG. 19, oxygen saturation StO2A in a case where there is no yellow coloring agent and oxygen saturation StO2B in a case where there is a yellow coloring agent are represented differently from each other. The oxygen saturation StO2B is shifted to be higher than the oxygen saturation StO2A in appearance due to the presence of the yellow coloring agent included in an image signal.

Thus, in order to be capable of accurately calculating the oxygen saturation even in the case of yellow coloring agent dependence as described above, the B3 image signal and the G3 image signal included in the third illumination light image are used in a case of correcting data used in calculating the oxygen saturation. The B3 image signal includes image information related to light transmitted through the B color filter BF in the third illumination light 58. The B3 image signal (specific color image signal) includes image information of the wavelength range B3, which has a sensitivity to a specific coloring agent other than hemoglobin, such as a yellow coloring agent (see FIG. 14). The B3 image signal has a certain sensitivity to the specific coloring agent, although the sensitivity to the specific coloring agent is not as high as that of the B1 image signal. Therefore, as shown in FIG. 20, the yellow coloring agent dependence of the B3 image signal is the degree of “medium” while the yellow coloring agent dependence of the B1 image signal is “high”. The oxygen saturation dependence of the B3 image signal is “low”, blood concentration dependence is “high”, and brightness dependence is “yes”.

Although the G3 image signal does not have as much sensitivity to the specific coloring agent as the B3 image signal, an image signal in a wavelength range G3 having a certain degree of sensitivity to the specific coloring agent is included (see FIG. 14). Therefore, the yellow coloring agent dependence of the G3 image signal is the degree of “low to medium”. The oxygen saturation dependence of the G3 image signal is “low”, blood concentration dependence is “high”, and brightness dependence is “yes”. In addition, since the yellow coloring agent dependence of the B2 image signal is “high”, the B2 image signal may be used instead of the B3 image signal in a case of correcting data used in calculating the oxygen saturation. The oxygen saturation dependence of the B2 image signal is “low”, blood concentration dependence is “high”, and brightness dependence is “yes”.

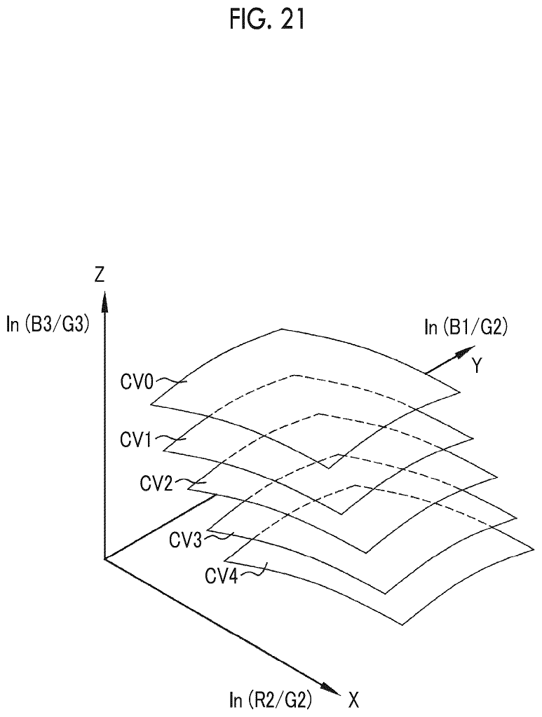

In a case where a relationship among the signal ratio ln (B1/G2) and the signal ratio ln (R2/G2), oxygen saturation, and a yellow coloring agent is represented by three-dimensional coordinates in which the signal ratio ln (R2/G2) is represented by an X-axis, the signal ratio ln (B1/G2) is represented by a Y-axis, and a signal ratio ln (B3/G3) is represented by a Z-axis, as shown in FIG. 21, curved surfaces CV0 to CV4 representing oxygen saturation are distributed in a Z-axis direction according to the coloring agent concentration of the yellow coloring agent. The curved surface CV0 represents oxygen saturation in a case where the concentration of the yellow coloring agent is “0” (no effect of the yellow coloring agent). The curved surfaces CVI to CV4 represent oxygen saturations in a case where the concentrations of the yellow coloring agent are “1” to “4”, respectively. It shows that the larger the number of the concentration is, the higher the concentration of the yellow coloring agent is. As shown by the curved surfaces CV0 to CV4, a value in the Z-axis direction changes to be lower as the concentration of the yellow coloring agent is higher.

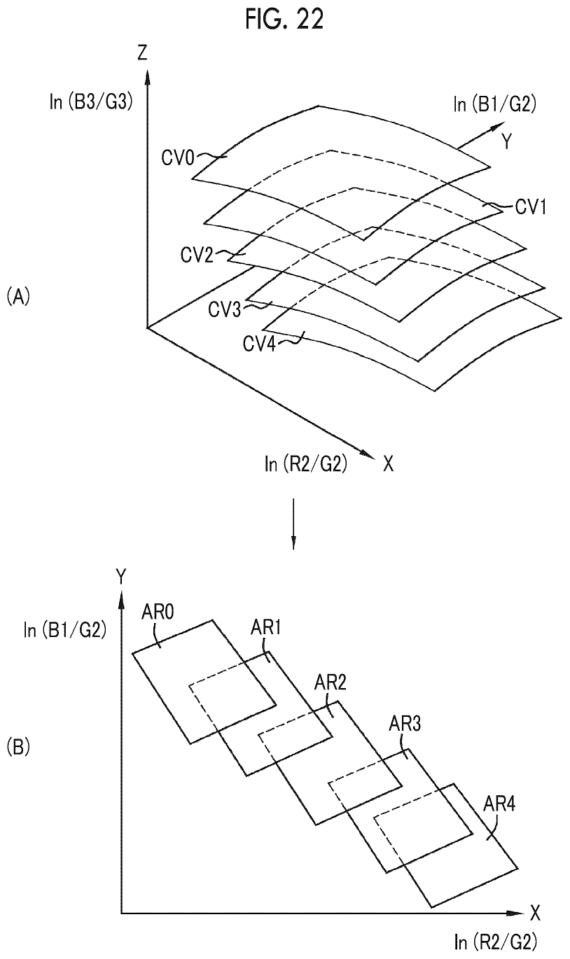

As shown in (A) of FIG. 22, in a case where the state of oxygen saturation expressed in three-dimensional coordinates of X, Y, and Z is expressed in two-dimensional coordinates of X and Y, as shown in (B) of FIG. 22, regions AR0 to AR4 representing the states of the oxygen saturation are distributed at different positions according to the concentrations of a yellow coloring agent, respectively. The regions AR0 to AR4 represent the distributions of the oxygen saturation in a case where the concentrations of the yellow coloring agent are “0” to “4”. By determining the contour line EL representing the oxygen saturation for each of the regions AR0 to AR4, the oxygen saturation corresponding to the concentration of the yellow coloring agent can be acquired (see FIG. 16). As shown in the regions AR0 to AR4, as the concentration of the yellow coloring agent increases, the value of the X-axis increases, and the value of the Y-axis decreases.

As shown in FIG. 23, the value of the X-axis (signal ratio ln (R2/G2)), the value of the Y-axis (signal ratio ln (B1/G2)), and the value of the Z-axis (signal ratio ln (B3/G3)) depend on a yellow coloring agent. The value of the X-axis (denoted as “X”) has “low to medium” yellow coloring agent dependence, the value of the Y-axis (denoted as “Y”) has “high” yellow coloring agent dependence, and the value of the Z-axis (denoted as “Z”) has “medium” yellow coloring agent dependence. In addition, the oxygen saturation dependence of the value of the Z-axis is “low to medium”, and blood concentration dependence thereof is “low to medium”. In addition, since the value of the Z axis is normalized by the G3 image signal, brightness dependence is “none”.

In the present embodiment, in a realistic case where an observation target is affected by a specific coloring agent such as a yellow coloring agent, selection of a plurality of pieces of data indicating oxygen saturation corresponding to the concentration of the yellow coloring agent in an endoscope image obtained by imaging the observation target is adopted as the correction of data used in calculating the oxygen saturation. Therefore, in this case, specifically, correcting data used in calculating the oxygen saturation in the correction mode means selecting any one of the curved surfaces CV0 to CV4 (see FIG. 21) representing the oxygen saturation.

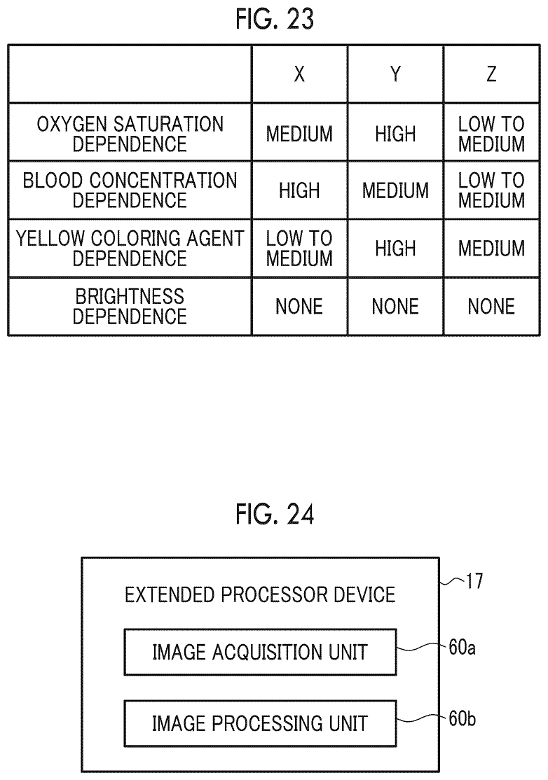

As shown in FIG. 24, the extended processor device 17 comprises an image acquisition unit 60a and an image processing unit 60b. The image acquisition unit 60a receives an image signal transmitted from the processor device 14 via the image communication unit 51. The image processing unit 60b performs image processing for generating an oxygen saturation image.

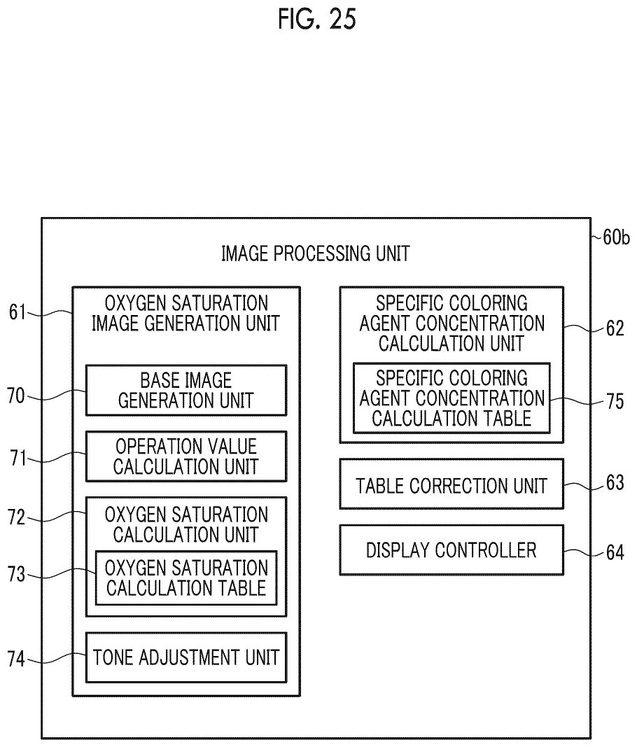

As shown in FIG. 25, the image processing unit 60b comprises an oxygen saturation image generation unit 61, a specific coloring agent concentration calculation unit 62, a table correction unit 63, and a display controller 64. In the extended processor device 17, a program related to various types of processing is incorporated in a program memory (not shown). As a central controller (not shown) composed of a processor executes the program in the program memory, functions of the oxygen saturation image generation unit 61, the specific coloring agent concentration calculation unit 62, the table correction unit 63, and the display controller 64 are realized.

The oxygen saturation image generation unit 61 comprises a base image generation unit 70, an operation value calculation unit 71, an oxygen saturation calculation unit 72, an oxygen saturation calculation table 73, and a tone adjustment unit 74. The base image generation unit 70 generates a base image based on an image signal from the processor device 14. The base image is used as a base of the oxygen saturation image OP. It is preferable that the base image is an image through which morphologic information such as the shape of an observation target can be ascertained. The base image is composed of the B2 image signal, the G2 image signal, and the R2 image signal. The base image may be a narrowband light image in which a blood vessel or a structure (ductal structure) is highlighted by narrowband light or the like.

The operation value calculation unit 71 calculates an operation value through operation processing based on the B1 image signal included in the oxygen saturation image signal, the G2 image signal, and the R2 image signal. Specifically, the operation value calculation unit 71 calculates a signal ratio B1/G2 between the B1 image signal and the G2 image signal and a signal ratio R2/G2 between the R2 image signal and the G2 image signal as the operation values used in the calculation of oxygen saturation. It is preferable that each of the signal ratio B1/G2 and the signal ratio R2/G2 is logarithmized (ln). In addition, color difference signals Cr and Cb, chroma saturation S, a color tone H, or the like calculated from the B1 image signal, the G2 image signal, and the R2 image signal may be used as the operation value.

The oxygen saturation calculation unit 72 calculates oxygen saturation based on an operation value with reference to the oxygen saturation calculation table 73. A correlation between the signal ratios B1/G2 and R2/G2, which are one of the operation values, and the oxygen saturation is stored in the oxygen saturation calculation table 73. In a case where the correlation is expressed in two-dimensional coordinates with the signal ratio ln (B1/G2) on a vertical axis and the signal ratio ln (R2/G2) on a lateral axis, the state of the oxygen saturation is expressed by the contour line EL extending in a lateral axis direction, and the contour line EL is distributed at different positions in a vertical axis direction in a case where the oxygen saturation is different (oxygen saturation calculation table 73a (see FIG. 16)). The oxygen saturation calculation table 73 includes the oxygen saturation calculation table 73a expressed in two-dimensional coordinates.

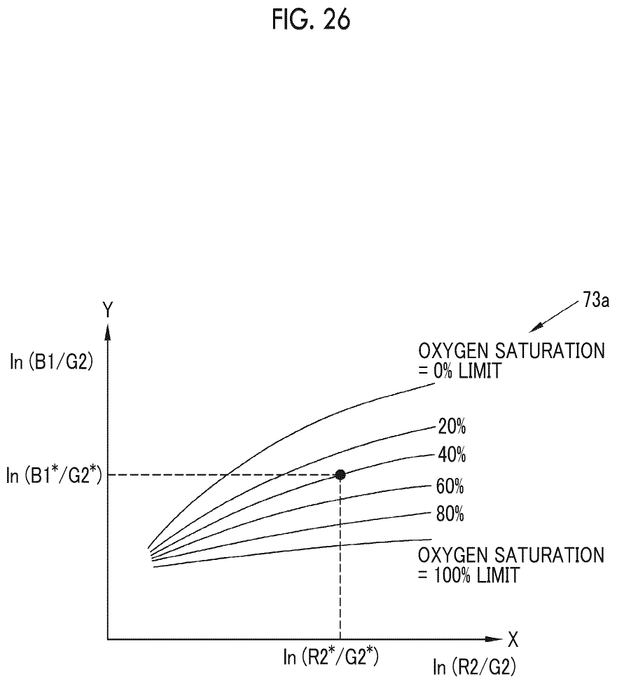

The oxygen saturation calculation unit 72 calculates oxygen saturation corresponding to the signal ratios B1/G2 and R2/G2 for each pixel with reference to the oxygen saturation calculation table 73. For example, as shown in FIG. 26, in a case where the signal ratio of a specific pixel is ln (B1*/G2*) and ln (R2*/G2*), the oxygen saturation corresponding to the signal ratio of ln (B1*/G2*) and ln (R2*/G2*) is “40%” with reference to the oxygen saturation calculation table 73a. Therefore, the oxygen saturation calculation unit 72 calculates the oxygen saturation of the specific pixel as “40%”.

The tone adjustment unit 74 generates an oxygen saturation image by performing composite color processing of changing the tone of a base image using oxygen saturation calculated by the oxygen saturation calculation unit 72. In the tone adjustment unit 74, in the base image, the tone is maintained for a region where the oxygen saturation exceeds a threshold value, and the tone is changed to a tone that changes according to the oxygen saturation for a region where the oxygen saturation is equal to or less than the threshold value. Accordingly, since only the tone of an abnormal part, in which oxygen saturation is equal to or lower than the threshold value, is changed while the tone of a normal part, in which oxygen saturation exceeds the threshold value, is maintained, the oxygen state of the abnormal part can be ascertained in a situation in which morphological information of the normal part can be observed.

In the tone adjustment unit 74, an oxygen saturation image may be generated through pseudo color processing of assigning a color corresponding to oxygen saturation regardless of the magnitude of the oxygen saturation. In a case of performing the pseudo color processing, a base image is not necessary.

The specific coloring agent concentration calculation unit 62 comprises a specific coloring agent concentration calculation table 75. The specific coloring agent concentration calculation unit 62 calculates a specific coloring agent concentration based on the specific coloring agent image signal including image information of a wavelength range, which has a sensitivity to a specific coloring agent other than blood hemoglobin, among coloring agents contained in an observation target, in the correction mode. Examples of the specific coloring agent include a yellow coloring agent such as bilirubin. It is preferable that the specific coloring agent image signal includes at least the B3 image signal. Specifically, the specific coloring agent concentration calculation unit 62 calculates the signal ratios ln (B1/G2), ln (G2/R2), and ln (B3/G3). The specific coloring agent concentration calculation unit 62 calculates specific coloring agent concentrations corresponding to the signal ratios ln (B1/G2), ln (G2/R2), and ln (B3/G3) with reference to the specific coloring agent concentration calculation table 75.

In the specific coloring agent concentration calculation table 75, a correlation between the signal ratios ln (B1/G2), ln (G2/R2), ln (B3/G3), and the specific coloring agent concentration is stored. For example, in a case where the ranges of the signal ratios ln (B1/G2), ln (G2/R2), and ln (B3/G3) are divided into five stages, specific coloring agent concentrations are associated with “0” to “4” with respect to the signal ratios ln (B1/G2), ln (G2/R2), and ln (B3/G3) in the ranges of the five stages, respectively, and are stored in the specific coloring agent concentration calculation table 75. It is preferable that the signal ratio B3/G3 is logarithmized (ln).

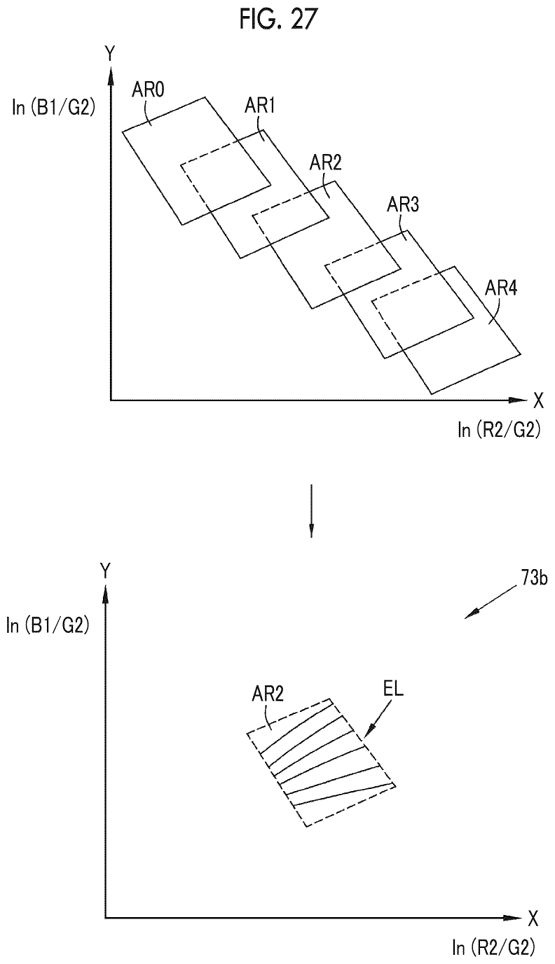

The table correction unit 63 performs table correction processing of correcting the oxygen saturation calculation table 73 based on a specific coloring agent concentration as the correction processing performed in the case of the correction mode. In the table correction processing, a correlation between the signal ratios B1/G2 and R2/G2 stored in the oxygen saturation calculation table 73 and the oxygen saturation is corrected. Specifically, in a case where the specific coloring agent concentration is “2”, the table correction unit 63 generates the contour line EL, which represents the state of the oxygen saturation, in the region AR2 where the specific coloring agent concentration corresponds to “2” among the regions AR0 to AR4 determined according to the specific coloring agent concentrations, as shown in FIG. 27. The table correction unit 63 generates a corrected oxygen saturation calculation table 73b by correcting the oxygen saturation calculation table 73 such that the generated contour line EL is generated.

In a case of correcting the oxygen saturation calculation table 73, which is data used in calculating the oxygen saturation, the table correction unit 63 sets a plurality of regions of interest in an acquired image and performs determination processing of determining whether or not the oxygen saturation calculation table 73 can be corrected using the regions of interest, with respect to each of the plurality of regions of interest. The oxygen saturation calculation table 73 is corrected using the region of interest for which it is determined that correction is possible based on the result of the determination processing. In the present embodiment, the oxygen saturation calculation table 73 is corrected using a specific coloring agent concentration.

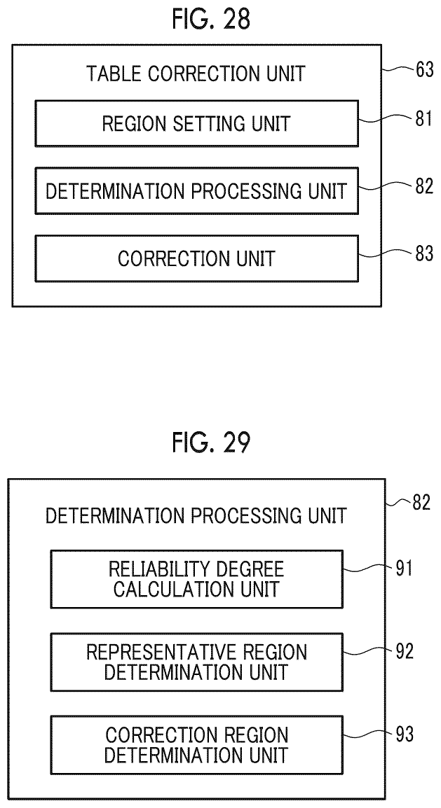

As shown in FIG. 28, the table correction unit 63 comprises a region setting unit 81, a determination processing unit 82, and a correction unit 83. The region setting unit 81 sets the plurality of regions of interest 11 in the endoscope image 10 acquired by the image acquisition unit 60a (see FIG. 1). In a case where the oxygen saturation calculation table 73 is corrected using the regions of interest 11, it is preferable to set the shapes, the areas, and the like of the regions of interest 11 in consideration of the fact that the oxygen saturation of a desired part in the endoscope image 10 can be more accurately calculated and in consideration of the calculation capacity and the like of the extended processor device 17. Since the setting of the regions of interest 11 is easier, it is preferable to set the regions of interest such that each of the plurality of regions of interest 11 has substantially the same shape, and it is preferable to set the regions of interest 11 such that each of the regions of interest 11 has substantially the same area. Here, the expression “substantially the same” includes both the same and almost the same.

For example, the region setting unit 81 sets the regions of interest 11 in the endoscope image 10 in a rectangular shape obtained by dividing the entire endoscope image 10 into 16 regions (see FIG. 1). Herein, the 16 regions of interest 11 have substantially the same shape and substantially the same area. The regions of interest 11 may be set with respect to a specific region, instead of the entire endoscope image 10. The specific region can be, for example, a vicinity of a center portion of the endoscope image 10, a portion of the endoscope image 10 other than an outer peripheral portion thereof, or the like.

The determination processing unit 82 performs determination processing of determining whether or not the oxygen saturation calculation table 73 can be corrected with respect to each of the plurality of regions of interest 11. As shown in FIG. 29, the determination processing unit 82 comprises a reliability degree calculation unit 91, a representative region determination unit 92, and a correction region determination unit 93.

In the determination processing, in a case of performing the parallel processing with respect to all of the plurality of regions of interest 11, the determination processing similarly proceeds with respect to all of the plurality of set regions of interest 11 each time the endoscope image 10 is acquired. In a case of performing the serial processing of processing one or a specific number of the plurality of regions of interest 11 in turn, one or a specific number of times of the determination processing is performed. In a case where the determination processing is completed, the determination processing proceeds in turn such that the next one or a specific number of times of the determination processing are performed, and the determination processing is completed with respect to the entire endoscope image 10 or the entire specific region.

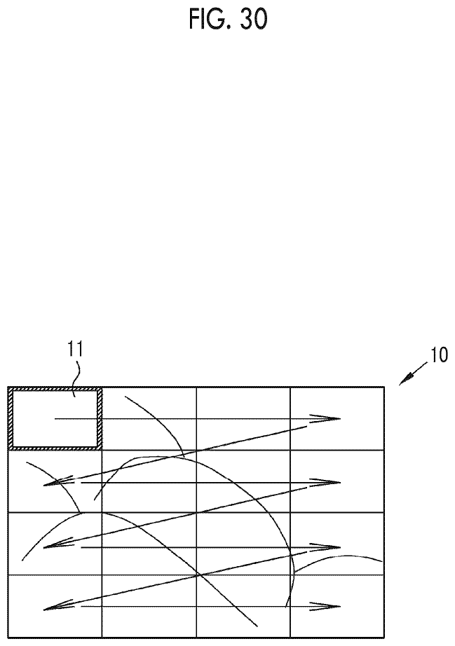

As shown in FIG. 30, in a case of performing the serial processing of processing the plurality of regions of interest 11 one by one in turn, the determination processing can be performed by moving the region of interest 11 with which the determination processing is performed so as to be shown by arrows, among the regions of interest 11 set with respect to the endoscope image 10, that is, by scanning the endoscope image 10 with the so-called regions of interest 11 in the order of the arrows. Regions of interest 11a each have one rectangular shape obtained by dividing the endoscope image 10 into 16 regions. In the drawing, the arrows indicate the order in which the determination processing is performed for the regions of interest 11.

Specifically, a method of first starting the determination processing of one of the plurality of regions of interest 11, performing next determination processing of another region of interest 11 after the determination processing of the region of interest 11 is completed, and repeating the determination processing until the determination processing of the entire endoscope image 10 is completed can be adopted.

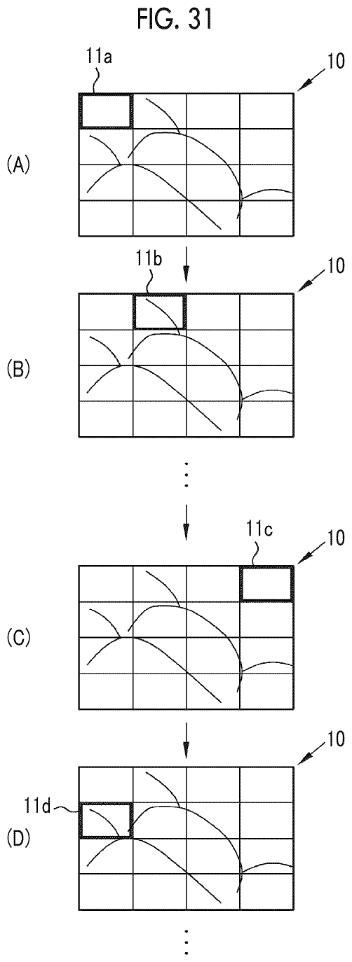

As shown in (A) of FIG. 31, for example, the region of interest 11a is set in the upper left of the endoscope image 10, and the determination processing is started. Next, as shown in (B) of FIG. 31, after the determination processing of the region of interest 11a is completed, the determination processing is performed with respect to a region of interest 11b, which is on the right of the region of interest 11a. In turn, the determination processing is performed with respect to a region of interest 11c on the right of the region of interest 11b, and the determination processing is performed with respect to a region of interest 11d at a left end of a second row of the endoscope image 10 as shown in (D) of FIG. 31 after the determination processing of the region of interest 11c set at an upper right end of the endoscope image 10 is completed as shown in (C) of FIG. 31. Hereinafter, the determination processing is performed in turn with respect to the regions of interest 11 in the same manner, and the determination processing set with respect to the endoscope image 10 can be completed by completing the determination processing with respect to all of the plurality of set regions of interest 11.

Since the determination processing is processing of determining whether or not the oxygen saturation calculation table 73 can be corrected with respect to each of the plurality of regions of interest 11, the determination processing is processing of setting an indicator related to appropriately obtaining oxygen saturation in a case where the oxygen saturation calculation table 73 is corrected using the regions of interest 11 and performing determination based on the indicator. In a case where a reliability degree set using image information of the regions of interest 11 is used as an indicator, the reliability degree is calculated for each region of interest 11 in the determination processing.

As a reliability degree, image information of each of a plurality of pixels included in one region of interest 11 can be used. The reliability degree may be calculated with respect to each pixel included in the region of interest 11, and an average of the reliability degrees of the plurality of pixels included in the region of interest 11 may be used as the reliability degree of the region of interest 11. It is preferable that the reliability degree affects the correction of data used in calculating oxygen saturation. One type of reliability degree may be calculated and used, or a plurality of types of reliability degrees may be calculated and used.

The reliability degree calculation unit 91 calculates at least one reliability degree that affects the correction of data used in calculating oxygen saturation for each pixel based on the B1 image signal, the G1 image signal, and the R1 image signal of each pixel included in the first illumination light image or on the B2 image signal, the G2 image signal, and the R2 image signal of each pixel included in the second illumination light image. The reliability degree is represented by, for example, a decimal between 0 and 1. The smaller the value of the reliability degree is, the lower the reliability degree is, and the larger the value of the reliability degree is, the higher the reliability degree is. In a case where the determination processing unit 82 calculates a plurality of types of reliability degrees, a final reliability degree of each pixel can be determined through a method of adopting a reliability degree having a minimum value among the plurality of types of reliability degrees, which is the method of adopting a reliability degree calculated using the plurality of types of reliabilities, or the like.

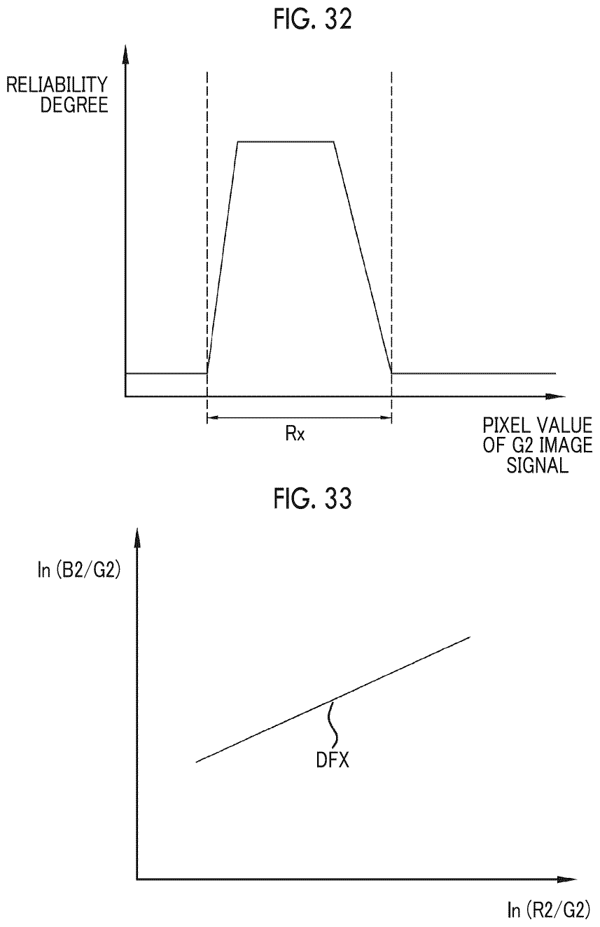

Examples of a factor that affects the correction of data used in calculating oxygen saturation include a pixel value in an endoscope image. Therefore, the pixel value can be used in calculating the reliability degree. As shown in FIG. 32, regarding the pixel value that affects the accuracy of correcting the data used in calculating oxygen saturation, the pixel value of the G2 image signal can be used. A reliability degree in a case where the pixel value of the G2 image signal is not within a certain range Rx tends to be lower than a reliability degree in a case where the pixel value of the G2 image signal is within the certain range Rx. The case where the pixel value is not within the certain range Rx is a case where the pixel value is a minimum pixel value, such as a dark portion, in addition to a case where the pixel value is a high pixel value, such as halation. In a case where the pixel value is not within the certain range Rx as described above, the accuracy of correcting the data used in calculating oxygen saturation tends to be low. Thus, the reliability degree can also be said to be accordingly low. The G1 image signal may be used in calculating the reliability degree using the pixel value, instead of the G2 image signal.

In addition, examples of the factor that affects the accuracy of correcting data used in calculating oxygen saturation include a disturbance. The disturbance includes at least the presence of bleeding, fat, residues, mucus, or residual liquids in an observation target. The reliability degree also fluctuates due to the disturbances. Regarding bleeding, which is one of the disturbances, as shown in FIG. 33, the reliability degree is determined according to a distance from a definition line DFX in a two-dimensional plane consisting of a vertical axis ln (B2/G2) and a lateral axis ln (R2/G2). Herein, the reliability degree is lower as coordinates of ln (B2/G2) and ln (R2/G2) plotted on the two-dimensional plane based on the B2 image signal, the G2 image signal, and the R2 image signal are positioned closer to the lower right. In addition, in a case where the coordinates are positioned in an upper left region with respect to the definition line DFX, the reliability degree depending on a degree of bleeding is set to a fixed value that is a high reliability degree. In FIG. 33, ln represents a natural logarithm. B2/G2 represents a signal ratio between the B2 image signal and the G2 image signal, and R2/G2 represents a signal ratio between the R2 image signal and the G2 image signal.

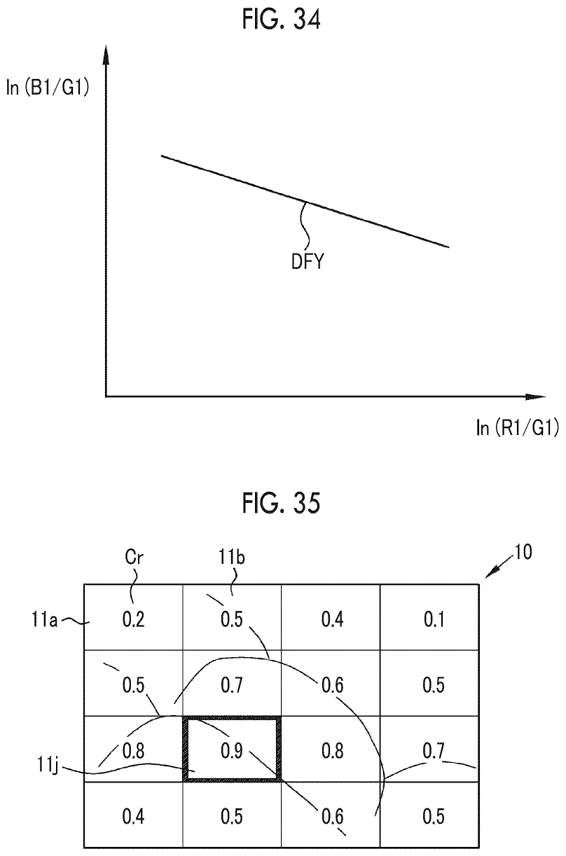

In addition, as shown in FIG. 34, regarding the fat, the residues, the residual liquids, or the mucus included in the disturbance, a reliability degree is determined according to a distance from a definition line DFY in a two-dimensional plane consisting of a vertical axis ln (B1/G1) and a lateral axis ln (R1/G1). Herein, the reliability degree is lower as coordinates of ln (B1/G1) and ln (R1/G1) plotted on the two-dimensional plane based on the B1 image signal, the G1 image signal, and the R1 image signal are positioned closer to the lower left. In addition, in a case where the coordinates are positioned in an upper right region with respect to the definition line DFY, the reliability degree depending on a degree of fat is set to a fixed value that is a high reliability degree. In FIG. 34, ln represents a natural logarithm. B1/G1 represents a signal ratio between the B1 image signal and the G1 image signal, and R1/G1 represents a signal ratio between the R1 image signal and the G1 image signal.

The reliability degree calculation unit 91 calculates a reliability degree for each pixel included in the region of interest 11 depending on one type or a plurality of types of the various types of reliability degrees. For example, in a case where one type of reliability degree is used, a reliability degree is selected from the reliability degree using the pixel value (see FIG. 32), the reliability degree considering bleeding (see FIG. 33), or the reliability degree considering the fat, the residues, the residual liquids, or the mucus (see FIG. 34). In addition, in a case where a plurality of reliability degrees are used, two or more types can be selected and used from the plurality of types of reliability degrees. The type of reliability degree may be selected based on a state of an observation target, a difference in a part, and the like.

It is preferable that the determination processing includes region reliability degree calculation processing of calculating a region reliability degree in each of the plurality of regions of interest 11. In the region reliability degree calculation processing, the region reliability degree, which is a reliability degree of each of the regions of interest 11, is calculated using the reliability degree for each pixel (hereinafter, referred to as a pixel reliability degree). The region reliability degree is calculated using the pixel reliability degree in pixels included in each of the plurality of regions of interest 11.

The region reliability degree can be acquired, for example, by arithmetically averaging the pixel reliability degrees of the respective pixels included in the region of interest 11. As the arithmetic average, a simple average, a weighted average, a trimmed average, or the like can be used. In a case where the simple average is used, a value obtained by dividing a value, which is obtained by summing a pixel reliability degree Pc in pixels included in the region of interest for all the pixels included in the region of interest 11, by the number of pixels N included in the region of interest can be used as a region reliability degree Rc. The relationship is represented by expression (1) below.

$$Region reliability degree Rc = \{ \sum ( Pixel reliability degree Pc of pixels included in region of interest ) \} \text{} / number of pixels N included in region of interest \qquad ( 1 )$$

The representative region determination unit 92 determines a representative region 85 from the plurality of regions of interest 11. The representative region determination unit 92 can perform representative region determination processing of determining a representative region by comparing the region reliability degrees Rc in the plurality of respective regions of interest 11 with each other after the region reliability degree Rc is calculated in each of the plurality of regions of interest 11. A region having the highest region reliability degree Rc can be determined as the representative region.

As shown in FIG. 35, the representative region 85 is determined by comparing the calculated region reliability degrees Rc in the plurality of respective regions of interest 11 with each other. In the example shown in FIG. 35, the value of the region reliability degree Rc calculated in each of the regions of interest 11 is shown by a numeral. The region reliability degree Rc in the region of interest 11a is 0.2, and the region reliability degree Rc in the region of interest 11b is 0.5. In the drawing, only some targets are assigned with reference numerals in some cases in order to avoid complication.

In the case shown in FIG. 35, in a case where the region reliability degrees Rc calculated in the plurality of respective regions of interest 11 are compared with each other, the region reliability degree Rc in a region of interest 11j is 0.9, which is the highest. Therefore, the representative region determination unit 92 determines the region of interest 11j as the representative region.

In a case where the region reliability degrees Re in the plurality of respective regions of interest 11 are the same value or there are a plurality of regions of interest 11 having a value within a range set in advance, all of the regions of interest 11 may be determined as the representative regions, or one region of interest 11, among the regions of interest 11, may be determined as the representative region based on a reference set in advance. Examples of the reference set in advance include a reference based on the position of the region of interest 11, such as determining the region of interest 11 close to the center of the endoscope image 10 as the representative region, a reference based on a specific type of reliability degree, such as a pixel value, a reference based on results of performing a weighted average, a trimmed average, or the like in addition to a simple average in calculating the reliability degree, and a reference based on other statistical values such as a median value and a mode value.

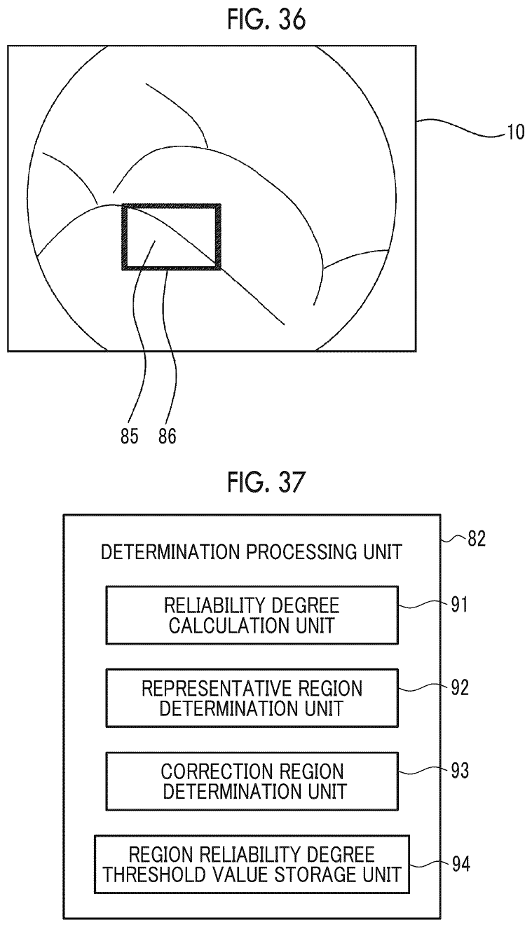

After determining the representative region 85, the representative region determination unit 92 displays the representative region in the endoscope image 10 on the extended display 18 in order to show the region for correcting the oxygen saturation calculation table to the user. As shown in FIG. 36, on the extended display 18, the representative region 85 is displayed by a display frame 86 representing the position and the shape of the region of interest 11 so that the representative region 85 is superimposed on the endoscope image 10. Until the representative region 85 is determined, the region of interest 11 is not displayed. In addition, in a case of performing setting and determination processing of the region of interest 11 described above with respect to each endoscope image 10 obtained for each frame, the position of the representative region 85 can be changed. In this case, since the display frame 86 indicating the representative region 85 also changes, the display frame 86 is displayed while moving like an animation in the endoscope image 10 with elapse of time in some cases.

By displaying the representative region 85 on the extended display 18, the user can easily ascertain at a glance at what position of the endoscope image 10 oxygen saturation can be more accurately calculated without effort.

The representative region determination unit 92 may determine the plurality of regions of interest 11 having the high region reliability degrees Rc as the representative region 85, in addition to determining a region having the highest region reliability degree Rc as the representative region 85. For example, the plurality of regions of interest 11 set in advance in descending order of the region reliability degree Rc may be used as the representative region 85. For example, three regions of interest 11 set in advance in descending order of the region reliability degree Rc may be used as the representative region 85. In this case, the region reliability degrees Rc for the plurality of regions of interest 11 set as the representative region 85 can be a value obtained by averaging the region reliability degrees Rc of the plurality of regions of interest 11.

The correction region determination unit 93 may determine the representative region 85 as a correction region that is the region of interest 11 with which the oxygen saturation calculation table can be corrected as it is. The correction region is the region of interest 11 for which it is determined that the correction of the oxygen saturation calculation table is possible, but the display controller 64 performs control of displaying the correction region on the extended display 18. Therefore, in the case shown in FIG. 35, the correction region determination unit 93 determines the representative region 85 as the correction region and displays the representative region 85 with the display frame 86 in the endoscope image 10. In addition, the correction unit 83 corrects the oxygen saturation calculation table using the correction region.

The correction region determination unit 93 may determine that the region of interest 11 determined to be the representative region 85 is the region of interest 11 with which the correction is possible after examining whether or not the oxygen saturation calculation table can be corrected using the region of interest 11. In this case, the correction region determination unit 93 determines whether or not the representative region 85 is the region of interest 11 with which the oxygen saturation calculation table can be corrected by comparing the region reliability degree Rc in the representative region 85 with a region reliability degree threshold value set in advance.

In this case, as shown in FIG. 37, the determination processing unit 82 may further include a region reliability degree threshold value storage unit 94. The region reliability degree threshold value storage unit 94 stores the region reliability degree threshold value for comparing with the region reliability degree Rc in the representative region 85 determined through the representative region determination processing. The correction region determination unit 93 performs processing of determining the correction region using the stored region reliability degree threshold value.

The region reliability degree threshold value storage unit 94 comprises a plurality of region reliability degree threshold values, and the correction region determination unit 93 sets a region reliability degree threshold value corresponding to the type of the region reliability degree Rc of the representative region 85 to be compared or the like, in advance. It is preferable that the corresponding region reliability degree threshold value is a threshold value for the reliability degree calculated through the same method as the region reliability degree Rc to be compared.

The correction region determination unit 93 compares the region reliability degree Rc in the region of interest 11j which is the representative region 85 with the set region reliability degree threshold value and determines the representative region 85 as the correction region in a case where the region reliability degree Rc is equal to or higher than the region reliability degree threshold value. On the other hand, in a case where the region reliability degree Rc is smaller than the region reliability degree threshold value, it is determined that the representative region 85 is an uncorrectable region with which the oxygen saturation calculation table cannot be corrected.

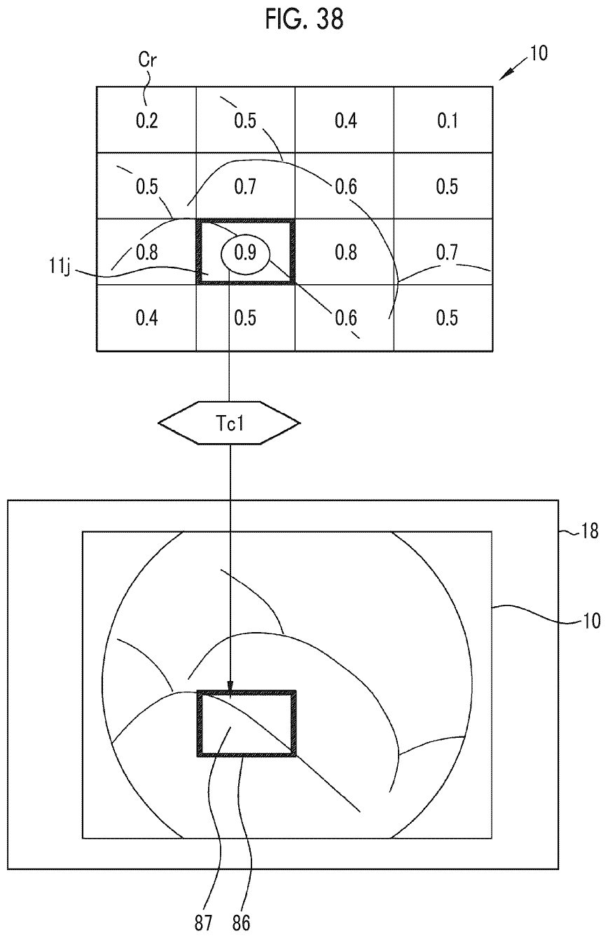

As shown in FIG. 38, the correction region determination unit 93 sets a region reliability degree threshold value Tc corresponding to the region reliability degree Rc in the region of interest 11 to 0.9 in advance (Tc1), and in a case where the region reliability degree Rc of the region of interest 11j, which is the representative region 85, is 0.9, the region reliability degree Rc of the region of interest 11j is equal to or higher than the region reliability degree threshold value Tc. Thus, the correction region determination unit 93 determines the region of interest 11j as a correction region 87. The correction region 87 is superimposed on the endoscope image 10 and is displayed by the display frame 86 on the extended display 18. In addition, the correction unit 83 corrects the oxygen saturation calculation table 73 using the correction region 87.

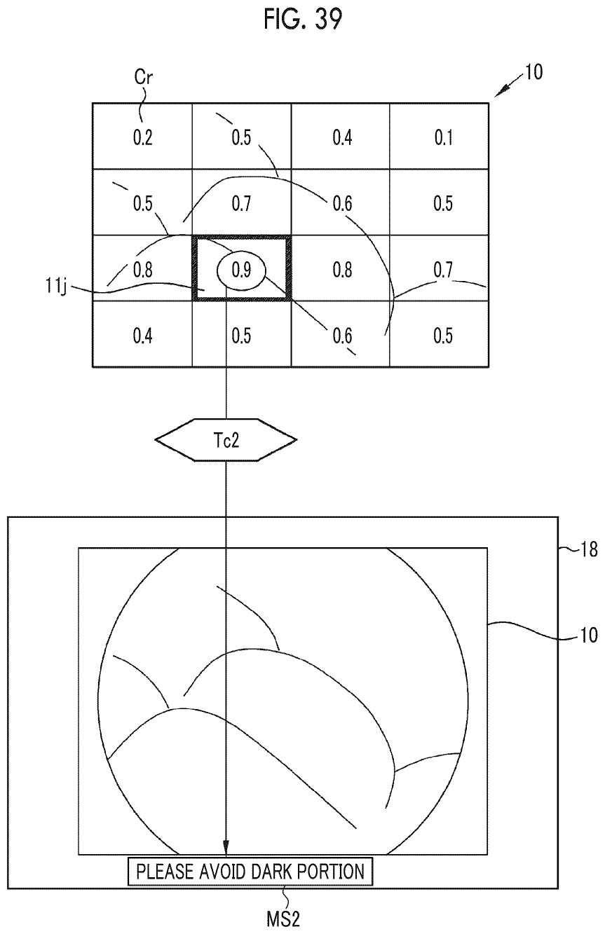

As shown in FIG. 39, the correction region determination unit 93 sets the region reliability degree threshold value Tc corresponding to the region reliability degree Rc in the region of interest 11 to 0.95 in advance (Tc2), and in a case where the region reliability degree Rc of the region of interest 11j, which is the representative region 85, is 0.9, the region reliability degree Rc of the region of interest 11j is lower than the region reliability degree threshold value Tc. Thus, the correction region determination unit 93 determines the region reliability degree Rc as an uncorrectable region. The uncorrectable region is not displayed on the extended display 18. In addition, in a case where there is no correction region 87, the correction unit 83 does not correct the oxygen saturation calculation table.

In addition, in a case where there is no region of interest 11 for which it is determined that the correction of the oxygen saturation calculation table 73 is possible, that is, in a case where there is no correction region 87, the correction region determination unit 93 may perform control of notifying the user of the fact that there is no region of interest 11 for which it is determined that the correction of the oxygen saturation calculation table 73 is possible. The notification to the user may be in any form that can be recognized by the user, may be other than display of text such as display on the display, and may be display of an icon or the like, notification by voice, or the like. As shown in FIG. 39, not displaying the display frame 86 indicating the correction region 87 is also one aspect of the notification to the user.

As another aspect of the notification to the user, as shown in FIG. 39, operation guidance MS2 for generating the correction region 87 may be displayed. The operation guidance MS2 can be guidance consisting of text or the like for generating the correction region 87 in the endoscope image 10. It is preferable that the content of the guidance is guidance for making a reliability degree in any of the regions of interest 11 in the endoscope image 10 equal to or higher than the region reliability degree threshold value Tc. For example, in a case where the reliability degree is based on a pixel value, it is preferable to display guidance for improving the pixel value in the endoscope image 10. The guidance may be made by displaying text or by displaying an icon or the like or by making a voice notification.

The correction unit 83 corrects the oxygen saturation calculation table 73 using the correction region 87. The oxygen saturation calculation table 73 is corrected using image information of the correction region 87. In the present embodiment, the oxygen saturation calculation table 73 is corrected by selecting any one of the curved surfaces CV0 to CV4 (see FIG. 21) representing oxygen saturation. Therefore, the correction unit 83 corrects the oxygen saturation calculation table 73 by selecting any one of the curved surfaces CV0 to CV4 (see FIG. 21) representing the oxygen saturation as described above using the image information of the correction region 87.

In addition, it is preferable that in a case where there is no region of interest 11 for which it is determined that the correction of the oxygen saturation calculation table 73 is possible, the oxygen saturation calculation unit 72 calculates the oxygen saturation of an observation target using the uncorrected oxygen saturation calculation table 73. Accordingly, the oxygen saturation can be more accurately calculated without performing undesirable correction with respect to the oxygen saturation calculation table 73.

Since the endoscope system 20 is configured as described above, even in a case where a region that is a problem related to the correction of the oxygen saturation calculation table is included in the endoscope image 10, the oxygen saturation calculation table 73 is corrected after identifying a region that is not a problem, a region that is most suitable for correction, and the like. Therefore, the oxygen saturation calculation table 73 can be more reliably corrected without excessive trial and error. In addition, the processing in the image processing unit 60b is, for example, automatically performed for each frame image, and the user does not need to perform an additional operation. Thus, the oxygen saturation calculation table 73 can be quickly corrected. Therefore, the endoscope system 20 can more reliably and quickly correct data used in calculating oxygen saturation.

The region setting unit 81 may set the regions of interest 11 having a shape other than a rectangle in the endoscope image 10. In addition, in the endoscope image 10, the regions of interest 11 having a shape and/or an area that is not constant may be set. In this case, it is preferable to set the regions of interest having shapes different from each other and/or having areas different from each other based on the positions of the region of interest 11 in the endoscope image 10.

In addition, the region setting unit 81 may set the regions of interest 11 such that at least two of the plurality of regions of interest 11 include pixels of the endoscope image 10 different from each other. In addition, it is preferable to set the regions of interest 11 such that at least two of the plurality of regions of interest 11 include the same pixel of the endoscope image 10. That is, in this case, a state where the regions of interest 11 overlap each other is caused.

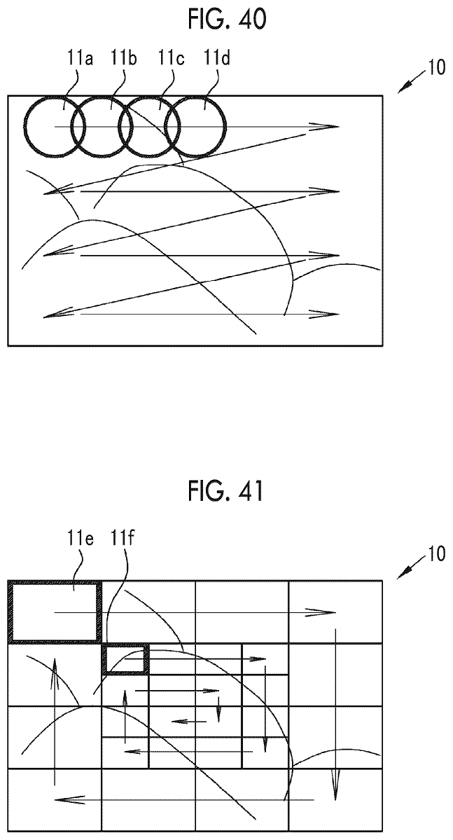

As shown in FIG. 40, the region setting unit 81 may set circular regions of interest 11a to 11d having a shape other than a rectangle. In order to avoid complication of the drawing, only some of the plurality of regions of interest 11 are shown. The circular regions of interest 11 are set to cover the entire endoscope image 10. The circular region of interest 11a is set to include the same pixels and to include pixels different from those of the region of interest 11b which is adjacent thereto, and the same applies to each of the regions of interest 11b to 11d. In a case of performing the serial processing of processing the plurality of regions of interest 11 one by one in turn, the determination processing can be performed by moving a target of the determination processing on the endoscope image 10 as shown by arrows, and by scanning the endoscope image 10 in the order of the arrows using the so-called regions of interest 11. Accordingly, the determination processing can be performed with respect to almost the entire endoscope image 10 while efficiently performing the determination processing.

As shown in FIG. 41, the region setting unit 81 may set rectangular regions of interest 11e and 11f that have the same rectangular shape but have areas different from each other. In order to avoid complication of the drawing, only a part of the display frame for displaying the region of interest 11 is shown. In the case of FIG. 41, the rectangle of the center portion of the endoscope image 10 is set to have a smaller area than the rectangle of a peripheral portion. In the endoscope image 10, peripheral darkening in which the amount of light in a peripheral portion of an image is insufficient occurs mainly due to a relationship with a light source or the like in some cases. By setting the rectangular regions of interest having different areas, it is possible to respond to a case where the center portion of the endoscope image 10, rather than the peripheral portion, can be adopted as the correction region 87.

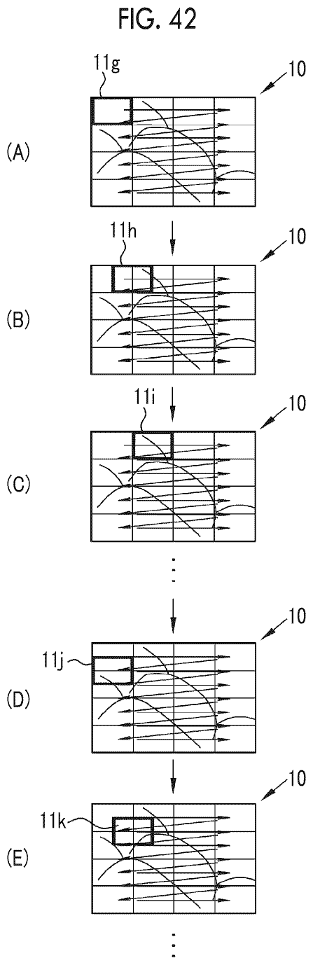

As shown in (A) of FIG. 42, the region setting unit 81 may set the plurality of regions of interest 11 in a state where the regions of interest 11 overlap each other. In a case of performing the serial processing, first, the determination processing unit 82 performs the determination processing with respect to a region of interest 11g, and then, as shown in (B) of FIG. 42, performs the determination processing with respect to a region of interest 11h. The region of interest 11g and the region of interest 11h overlap each other in a range of approximately ½ of the long side of the rectangle. Next, as shown in (C) of FIG. 42, the determination processing is performed with respect to the region of interest 11h and a region of interest 11i that overlap each other in a range of approximately ½ of the long side of the rectangle. Similarly, after the determination processing of the region of interest 11 having the right end of the endoscope image is completed, as shown in (D) of FIG. 42, the determination processing is performed with respect to the region of interest 11j that overlaps the region of interest 11g in a range of approximately ½ of the short side, and then, as shown in (E) of FIG. 42, the determination processing is performed with respect to a region of interest 11k that overlaps the regions of interest 11g and 11h in a range of approximately ½ of the long side and that overlaps the region of interest 11j in a range of approximately ½ of the short side. Hereinafter, the determination processing similarly proceeds in the order indicated by the arrows.

In the case of the parallel processing, the determination processing is performed at once with respect to all of the plurality of regions of interest 11 set in the endoscope image 10, such as the regions of interest 11g, 11h, 11i, 11j, and 11k. In a case where the determination processing unit 82 performs the determination processing of the regions of interest 11 through the parallel processing, the determination processing unit 82 performs the determination processing in parallel at once with respect to each of the plurality of regions of interest 11 set in this manner. By setting the regions of interest 11 such that the regions of interest 11 overlap each other in this manner, the determination processing can be more precisely performed with respect to almost the entire endoscope image 10 while efficiently performing the determination processing.

The region setting unit 81 may set the regions of interest 11 in a part of the endoscope image 10. In addition, even in a case where the region setting unit 81 sets the regions of interest 11 in the entire endoscope image 10, the determination processing unit 82 may perform the determination processing on some of the plurality of regions of interest 11 set by the region setting unit 81. Some of the plurality of regions of interest 11 can be set in any manner according to the type of the endoscope image 10, the type of an observation target, and the like. For example, it is preferable to set some of the plurality of regions of interest 11 as the center portion of the endoscope image 10. Accordingly, the determination processing can be efficiently performed in consideration of a case where peripheral darkening occurs in the endoscope image 10 and the like.

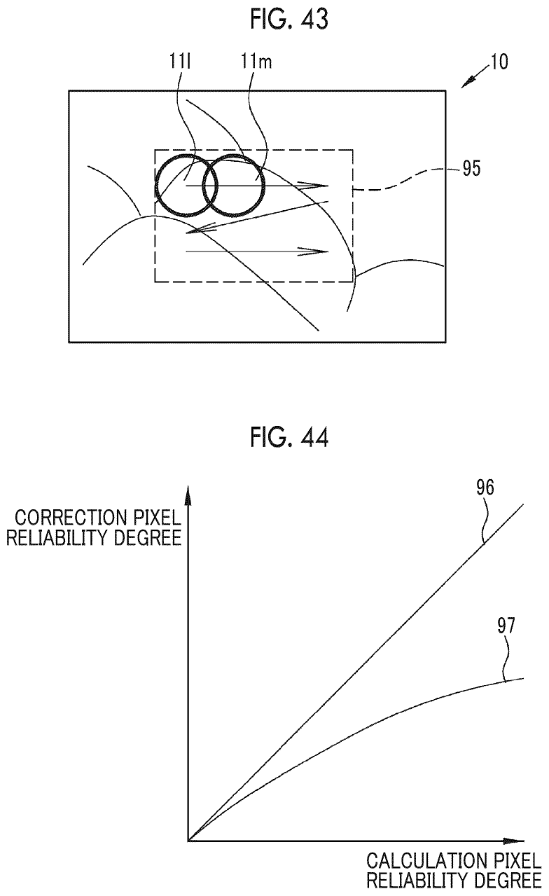

As shown in FIG. 43, in a case where the region setting unit 81 sets the regions of interest 11 as in the case of FIG. 41, the determination processing unit 82 may perform the determination processing on the regions of interest 11 included in a center region 95 of the endoscope image 10 and may not perform the determination processing on the regions of interest 11 other than the center region 95.

In addition, the determination processing unit 82 may complete the determination processing with respect to the endoscope image 10 by performing the determination processing one at a time for each of all the plurality of regions of interest 11 set by the region setting unit 81 with respect to the endoscope image 10 or may complete the determination processing by performing the determination processing a plurality of times on some or all of the plurality of regions of interest 11. In addition, in each of the plurality of times of the determination processing, the region setting unit 81 may set the regions of interest 11 having shapes and/or areas different from each other or may set the regions of interest 11 having the same shape and/or area. Accordingly, the determination processing can be more precisely and efficiently performed according to the type of the endoscope image 10 or an observation target, the purpose of correcting the oxygen saturation calculation table, and the like.

The reliability degree calculation unit 91 may correct a pixel reliability degree according to the position of each pixel in the endoscope image 10. The pixel reliability degree which is corrected will be referred to as a corrected pixel reliability degree. In a case where the pixel reliability degree is corrected, the reliability degree calculation unit 91 calculates a region reliability degree using the corrected pixel reliability degree. As a correction method corresponding to the position of each pixel in the endoscope image 10, correction corresponding to a distance of each pixel from the center of the endoscope image 10 is preferable. In a case where a lateral axis of the endoscope image 10 is defined as x and a vertical axis thereof is defined as y, the distance can be calculated by a square root of a sum of squares of a difference between a value of an x-axis and a value of a y-axis of the coordinate of the center of the endoscope image 10 and a value of the x-axis and a value of the y-axis of a coordinate of interest.

For a certain pixel that is a calculation target of the pixel reliability degree, the pixel is divided into, for example, two types including “short range” and “long range” according to a distance from the center of the endoscope image 10, and the pixel reliability can be corrected using correction data pieces different from each other. The correction data is set in advance by investigating a relationship between pixel reliability degrees of a plurality of pixels having different positions in the endoscope image 10 and the oxygen saturation in advance. In addition, in this case, standards for classifying into the “short range” pixel and the “long range” pixel are also set together with the creation of the correction data.

As shown in FIG. 44, the correction data indicates a relationship between a calculation pixel reliability degree which is a pixel reliability degree before correction and a correction pixel reliability degree. For example, short range correction data 96 is data in which the pixel reliability before correction and the corrected pixel reliability degree are in a proportional relationship regardless of the magnitude of the pixel reliability degree before correction. On the other hand, long range correction data 97 is correction data in which an increase in the correction pixel reliability degree is suppressed in a case where the pixel reliability degree before correction is larger than in a case where the pixel reliability degree before correction is smaller.

By using the corrected pixel reliability degree, the region reliability degree Rc can be more preferably calculated, and the accuracy of finally obtained oxygen saturation can be improved.

The reliability degree calculation unit 91 may calculate a region reliability degree after performing weighting according to the area of the region of interest 11 in the region reliability degree calculation processing. In addition, in the region reliability degree calculation processing, the reliability degree calculation unit 91 may calculate the region reliability degree after performing weighting according to a distance of the region of interest 11 from the center of the endoscope image 10. The distance of the region of interest 11 from the center of the endoscope image 10 can be a distance between the center or centroid of the region of interest 11 and the center of the endoscope image 10 (hereinafter, referred to as a region distance). In a case where a lateral axis of the endoscope image 10 is defined as x and a vertical axis thereof is defined as y, the region distance can be calculated by a square root of a sum of squares of a difference between a value of an x-axis and a value of a y-axis of the coordinate of the center of the endoscope image 10 and a value of the x-axis and a value of the y-axis of the center or centroid of the region of interest 11 in the x-axis and the y-axis.

The amount of the weight is determined by weighting data indicating a relationship between the area or the region distance of the region of interest 11 and the amount of the weight. The weighting data is set by investigating in advance a relationship between the area or the region distance of the region of interest 11 and oxygen saturation.

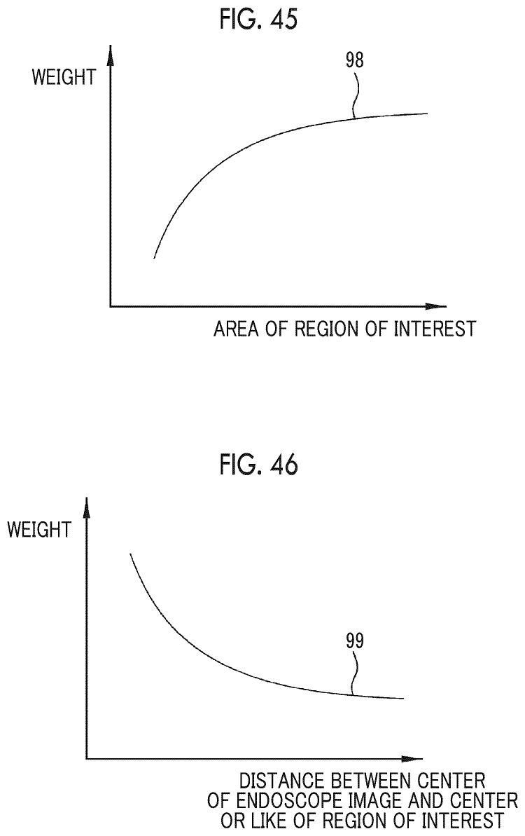

As shown in FIG. 45, weighting data related to an area 98 of the region of interest 11 indicates a relationship between the area and the weight of the region of interest 11. For example, the weighting data related to the area 98 indicates a relationship in which the larger the area of the region of interest 11 is, the larger the weighting amount is, and is data in which the proportion of the weighting amount is increased in a range where the area of the region of interest 11 is smaller than in a range where the area is larger.

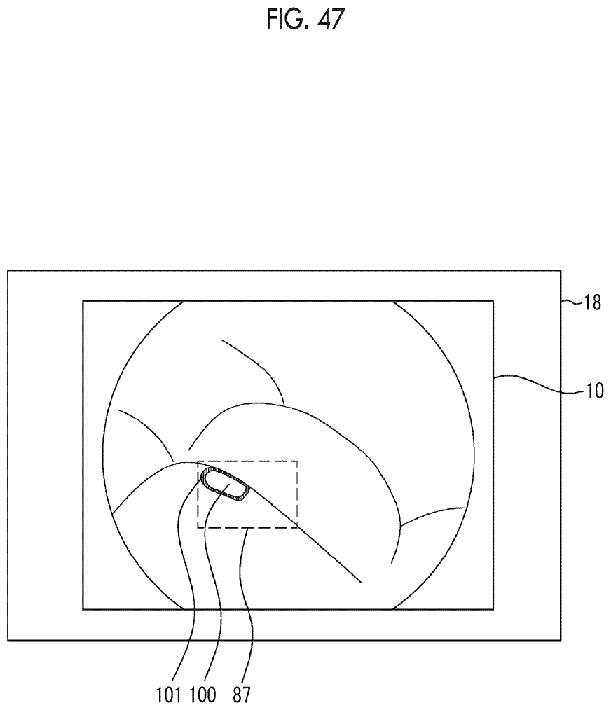

As shown in FIG. 46, weighting data related to a region distance 99 indicates a relationship between the region distance and the weight. For example, the weighting data related to the region distance 99 indicates a relationship in which the larger the distance is, the smaller the weighting amount is, and is data in which the proportion of the weighting amount is decreased in a range where the region distance is smaller than in a range where the region distance is larger.

In addition, it is preferable that the reliability degree calculation unit 91 performs both weighting according to the area of the region of interest 11 and weighting according to the region distance in the region reliability degree calculation processing. Therefore, the weighted region reliability degree Rc is obtained by multiplying the calculated region reliability degree Rc by a weight corresponding to the area of the region of interest 11 and a weight corresponding to the region distance.

In a case where the plurality of regions of interest 11 having the high region reliability degrees Rc are set as the representative region 85 and also in a case where an average value of the region reliability degrees Rc of the plurality of regions of interest 11 is set as the region reliability degree Rc of the representative region 85, it is preferable that the representative region determination unit 92 calculates the average value of the region reliability degrees Rc after performing weighting with respect to the region reliability degrees Rc.

By performing weighting with respect to the region reliability degree Rc, the region reliability degree Rc can be more preferably calculated, and the accuracy of finally obtained oxygen saturation can be improved.

The display controller 64 performs control of displaying the correction region 87 on the extended display 18. However, in addition to displaying the region of the correction region 87 on the endoscope image 10 in a superimposed manner by using the display frame 86 or the like, the display controller 64 may perform control of displaying, on the extended display 18, a pixel that has a pixel reliability degree which is equal to or higher than a pixel reliability degree threshold value set in advance, among pixels included in the correction region 87, in an aspect of being distinguishable from other pixels. Accordingly, a region on the endoscope image 10, for which it is determined that the correction of the oxygen saturation calculation table 73 is possible, can be understood in detail in units of pixels or the like.

As shown in FIG. 47, a pixel that is included in the correction region 87 and that has an image reliability degree equal to or higher than the pixel reliability degree threshold value set in advance may be displayed in a color that can be differentiated from other pixels. The display may be performed by coloring each pixel. In order to make it easy to recognize, a correction pixel group 100, in which a pixel having a pixel reliability degree equal to or higher than the pixel reliability degree threshold value is combined with several pixels around that pixel, can be displayed by an outline 101 indicating the region of the correction pixel group 100 by coloring the region of the correction pixel group 100.

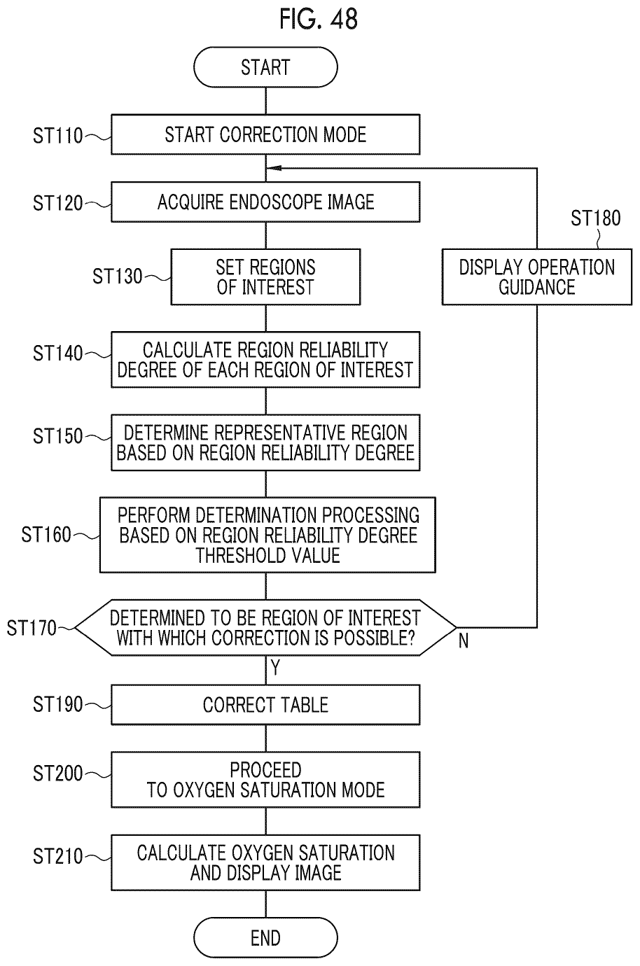

The series of procedures of the processing in the endoscope system or the like will be described using a flowchart. As shown in FIG. 48, in the endoscope system 20, the correction mode is started in a case of calculating oxygen saturation of an observation target (step ST110). The image acquisition unit 60a acquires the endoscope image 10 obtained by imaging the observation target with the endoscope 12 (step ST120). The region setting unit 81 sets the regions of interest 11 in the endoscope image 10 (step ST130).

The determination processing unit 82 performs the determination processing for determining whether or not each region of interest 11 is a region of interest 11 with which correction is possible. In the determination processing, the region reliability degree Rc of each region of interest 11 is calculated (step ST140), the representative region 85 is determined based on the calculated region reliability degree Rc (step ST150), and the determination processing of comparing the region reliability degree Rc in the representative region 85 with the region reliability degree threshold value is performed (step ST160). In a case where the region reliability degree Rc is equal to or higher than the region reliability degree threshold value, it is determined that the representative region 85 is the correction region 87 that is the region of interest 11 with which correction is possible (Y in step ST170).

In a case where the region reliability degree Rc is lower than the region reliability degree threshold value (N in step ST170), the operation guidance MS2 for generating the correction region 87 on the extended display 18 is displayed by text (step ST180), the user is prompted to obtain an appropriate endoscope image, and the procedures from the acquisition of the endoscope image (step ST120) are performed again.

The oxygen saturation calculation table is corrected using the correction region 87 (step ST190). In a case where the correction of the oxygen saturation calculation table is completed, the processing proceeds to the oxygen saturation mode (step ST200), the oxygen saturation of the endoscope image 10 acquired in a case where the processing proceeds to the oxygen saturation mode is calculated, and a generated oxygen saturation image is displayed (step ST210).

The function of the extended processor device 17 may be configured to be exerted by the processor device 14, or the function of the extended processor device 17 may be configured to be exerted by both the extended processor device 17 and the processor device 14. Therefore, as the processor device having the processor that corrects the oxygen saturation calculation table to generate the oxygen saturation image, only the processor device 14, only the extended processor device 17, or both the processor device 14 and the extended processor device 17 can be used.

In the embodiment, hardware structures of processing units that perform various types of processing such as the processor device 14, including the DSP 45, the image processing unit 50, the image communication unit 51, the display controller 52, and the central controller 53, and the extended processor device 17, including the image acquisition unit 60a and the image processing unit 60b, are various types of processors as described below. The various types of processors include a central processing unit (CPU) that is a general-purpose processor which executes software (program) and which functions as various types of processing units, a programmable logic device (PLD) that is a processor of which a circuit configuration can be changed after manufacturing a graphics processing unit (GPU), a field-programmable gate array (FPGA), and the like, and a dedicated electric circuit that is a processor having a dedicated circuit configuration designed to execute various types of processing.

One processing unit may be composed of one of the various types of processors or may be composed of a combination of two or more processors of the same type or different types (for example, a combination of a plurality of FPGAs, a combination of a CPU and an FPGA, or a combination of a CPU and a GPU). In addition, one processor may constitute a plurality of processing units. As an example in which one processor constitutes a plurality of processing units, in multiple cores, there is a form in which one processor is configured by a combination of one or more CPUs and software and the processor functions as the plurality of processing units, as represented by a computer such as a client and a server. In a single core, there is a form in which, as represented by a system on a chip (SoC) and the like, a processor that realizes functions of the entire system including a plurality of processing units with one integrated circuit (IC) chip is used. As described above, the various types of processing units are composed of one or more of the various types of processors used as a hardware structure.

Further, the hardware structures of the various types of processors are, more specifically, an electric circuit (circuitry) in a form in which circuit elements such as semiconductor elements are combined. In addition, a hardware structure of a storage unit is a storage device such as a hard disc drive (HDD) and a solid-state drive (SSD).

From the above description, the following supplementary notes can be understood.

### Supplementary Note 1

An endoscope system that calculates oxygen saturation of an observation target using data used in calculating the oxygen saturation, the endoscope system comprising:
a processor,
in which the processor is configured to:
acquire an image obtained by imaging the observation target;
set a plurality of regions of interest in the image;
perform determination processing of determining, with respect to each of the plurality of regions of interest, whether or not correction of the data using the region of interest is possible; and
correct the data using the region of interest for which it is determined that the correction of the data is possible.

### Supplementary Note 2

The endoscope system according to supplementary note 1,
in which the determination processing includes region reliability degree calculation processing of calculating a region reliability degree of each of the plurality of regions of interest based on the region of interest and representative region determination processing of determining a representative region by comparing the region reliability degrees of the plurality of respective regions of interest with each other.

### Supplementary Note 3

The endoscope system according to supplementary note 2,
in which the region reliability degree is calculated using a pixel reliability degree calculated for each pixel included in each of the plurality of regions of interest.

### Supplementary Note 4

The endoscope system according to supplementary note 2 or 3,
in which the determination processing includes region reliability degree comparison processing of comparing the region reliability degree of the representative region determined through the representative region determination processing with a region reliability degree threshold value set in advance.

### Supplementary Note 5

The endoscope system according to supplementary note 3 or 4,
in which the processor is configured to:
correct the pixel reliability degree according to a position of each pixel in the image, and
the region reliability degree is calculated using the corrected pixel reliability degree.

### Supplementary Note 6

The endoscope system according to any one of supplementary notes 2 to 5,
in which in the region reliability degree calculation processing, the region reliability degree is calculated after performing weighting according to an area of the region of interest.

### Supplementary Note 7

The endoscope system according to any one of supplementary notes 2 to 6,
in which in the region reliability degree calculation processing, the region reliability degree is calculated after performing weighting according to a distance of the region of interest from a center of the image.

### Supplementary Note 8

The endoscope system according to any one of supplementary notes 1 to 7,
in which the processor is configured to:
set the regions of interest such that each of the plurality of regions of interest has substantially the same shape.

### Supplementary Note 9

The endoscope system according to any one of supplementary notes 1 to 8,
in which the processor is configured to:
set the regions of interest such that each of the plurality of regions of interest has substantially the same area.

### Supplementary Note 10

The endoscope system according to any one of supplementary notes 1 to 9,
in which the processor is configured to:
set the regions of interest that have shapes different from each other and/or areas different from each other based on positions of the regions of interest in the image.

### Supplementary Note 11

The endoscope system according to any one of supplementary notes 1 to 10,
in which the processor is configured to:
set the regions of interest such that at least two of the plurality of regions of interest include the same pixel of the image.

### Supplementary Note 12

The endoscope system according to any one of supplementary notes 1 to 11,
in which the processor is configured to:
set the regions of interest such that each of the plurality of regions of interest includes pixels of the image different from each other.

### Supplementary Note 13

The endoscope system according to any one of supplementary notes 1 to 12,
in which the processor is configured to:
perform control of giving a notification to a user in a case where there is no region of interest for which it is determined that the correction of the data is possible.

### Supplementary Note 14

The endoscope system according to any one of supplementary notes 1 to 13,
in which the processor is configured to:
perform control of displaying the region of interest for which it is determined that the correction of the data is possible on a display.

### Supplementary Note 15

The endoscope system according to any one of supplementary notes 3 to 14,
in which the processor is configured to:
perform control of displaying a pixel having a pixel reliability degree equal to or higher than a pixel reliability degree threshold value set in advance on a display in an aspect of being distinguishable from other pixels.

### Supplementary Note 16

The endoscope system according to any one of supplementary notes 1 to 15,
in which the processor is configured to:
calculate, in a case where the data is corrected, the oxygen saturation of the observation target using the corrected data.

### Supplementary Note 17

The endoscope system according to any one of supplementary notes 1 to 16,
in which the processor is configured to:
calculate, in a case where there is no region of interest for which it is determined that the correction of the data is possible, the oxygen saturation of the observation target using the uncorrected data.

### Supplementary Note 18

The endoscope system according to any one of supplementary notes 1 to 17,
in which the processor is configured to:
correct the data using image information of the region of interest for which it is determined that the correction of the data is possible.

### Supplementary Note 19

The endoscope system according to supplementary note 18,
in which the image information is a specific coloring agent concentration of a specific coloring agent other than blood hemoglobin.

### Supplementary Note 20

The endoscope system according to supplementary note 19,
in which the specific coloring agent is a yellow coloring agent.

### EXPLANATION OF REFERENCES

10: endoscope image
11, 11a to 11m: region of interest
12: endoscope
12a: insertion part
12b: operating part
12c: mode switching switch
12d: zoom operation switch
13: light source device
14: processor device
15: display
16: user interface
17: extended processor device
18: extended display
20: endoscope system
21: light source unit
21a: V-LED
21b: BS-LED
21c: BL-LED
21d: G-LED
21e: R-LED
22: light source processor
23: optical path combining unit
24: light guide
30: illumination optical system
31: imaging optical system
32: illumination lens
35: objective lens
36: imaging sensor
37: imaging processor
40: CDS/AGC circuit
41: A/D converter
45: DSP
50: image processing unit
51: image communication unit
52: display controller
53: central controller
55: white light
56: first illumination light
57: second illumination light
58: third illumination light
55a, 55b, 56a, 56b: curve
60a: image acquisition unit
60b: image processing unit
61: oxygen saturation image generation unit
62: specific coloring agent concentration calculation unit
63: table correction unit
64: display controller
70: base image generation unit
71: operation value calculation unit
72: oxygen saturation calculation unit
73, 73a: oxygen saturation calculation table
74: tone adjustment unit
75: specific coloring agent concentration calculation table
81: region setting unit
82: determination processing unit
83: correction unit
85: representative region
86: display frame
87: correction region
91: reliability degree calculation unit
92: representative region determination unit
93: correction region determination unit
94: region reliability degree threshold value storage unit
95: center region
96: short range correction data
97: long range correction data
98: weighting data related to area
99: weighting data related to region distance
100: correction pixel group
101: outline
NP1: white light image
NP2: white light equivalent image
OP oxygen saturation image
BF: B color filter
GF: G color filter
RF: R color filter
CV0 to CV4: curved surface
AR0 to AR4: region
MS1: message
MS2: operation guidance
DFX: definition line
DFY: definition line
Tc1, Tc2: region reliability degree threshold value
ST110 to ST210: step

What is claimed is:

1. An endoscope system that calculates oxygen saturation of an observation target using data used in calculating the oxygen saturation, the endoscope system comprising:
a processor,
wherein the processor is configured to:
acquire an image obtained by imaging the observation target;
set a plurality of regions of interest in the image;
perform determination of, with respect to each of the plurality of regions of interest, whether or not correction of the data using the region of interest is possible; and
correct the data using the region of interest for which it is determined that the correction of the data is possible.

2. The endoscope system according to claim 1,
wherein the determination processing includes region reliability degree calculation processing of calculating a region reliability degree of each of the plurality of regions of interest based on the region of interest and representative region determination processing of determining a representative region by comparing the region reliability degrees of the plurality of respective regions of interest with each other.

3. The endoscope system according to claim 2,
wherein the region reliability degree is calculated using a pixel reliability degree calculated for each pixel included in each of the plurality of regions of interest.

4. The endoscope system according to claim 2,
wherein the determination processing includes region reliability degree comparison processing of comparing the region reliability degree of the representative region determined through the representative region determination processing with a region reliability degree threshold value set in advance.

5. The endoscope system according to claim 3,
wherein the processor is configured to:
correct the pixel reliability degree according to a position of each pixel in the image, and
the region reliability degree is calculated using the corrected pixel reliability degree.

6. The endoscope system according to claim 2,
wherein in the region reliability degree calculation processing, the region reliability degree is calculated after performing weighting according to an area of the region of interest.

7. The endoscope system according to claim 2,
wherein in the region reliability degree calculation processing, the region reliability degree is calculated after performing weighting according to a distance of the region of interest from a center of the image.

8. The endoscope system according to claim 1,
wherein the processor is configured to:
set the regions of interest such that each of the plurality of regions of interest has substantially the same shape.

9. The endoscope system according to claim 1,
wherein the processor is configured to:
set the regions of interest such that each of the plurality of regions of interest has substantially the same area.

10. The endoscope system according to claim 1,
wherein the processor is configured to:
set the regions of interest that have shapes different from each other and/or areas different from each other based on positions of the regions of interest in the image.

11. The endoscope system according to claim 1,
wherein the processor is configured to:
set the regions of interest such that at least two of the plurality of regions of interest include the same pixel of the image.

12. The endoscope system according to claim 1,
wherein the processor is configured to:
set the regions of interest such that each of the plurality of regions of interest includes pixels of the image different from each other.

13. The endoscope system according to claim 1,
wherein the processor is configured to:
perform control of giving a notification to a user in a case where there is no region of interest for which it is determined that the correction of the data is possible.

14. The endoscope system according to claim 1,
wherein the processor is configured to:
perform control of displaying the region of interest for which it is determined that the correction of the data is possible on a display.

15. The endoscope system according to claim 3,
wherein the processor is configured to:
perform control of displaying a pixel having a pixel reliability degree equal to or higher than a pixel reliability degree threshold value set in advance on a display in an aspect of being distinguishable from other pixels.

16. The endoscope system according to claim 1,
wherein the processor is configured to:
calculate, in a case where the data is corrected, the oxygen saturation of the observation target using the corrected data.

17. The endoscope system according to claim 1,
wherein the processor is configured to:
calculate, in a case where there is no region of interest for which it is determined that the correction of the data is possible, the oxygen saturation of the observation target using the uncorrected data.

18. The endoscope system according to claim 1,
wherein the processor is configured to:
correct the data using image information of the region of interest for which it is determined that the correction of the data is possible.

19. The endoscope system according to claim 18,
wherein the image information is a concentration of a specific coloring agent other than blood hemoglobin.

20. The endoscope system according to claim 19,
wherein the specific coloring agent is a yellow coloring agent.

21. An image processing apparatus that calculates oxygen saturation of an observation target, the image processing apparatus comprising:
a processor,
wherein the processor is configured to:
acquire an image obtained by imaging the observation target;
set a plurality of regions of interest in the image;
perform determination of, with respect to each of the plurality of regions of interest, whether or not correction of data using the region of interest is possible; and
correct the data using the region of interest for which it is determined that the correction of the data is possible.

22. An operation method of an endoscope system of calculating oxygen saturation of an observation target, the operation method comprising:
a step of acquiring an image obtained by imaging the observation target;
a step of setting a plurality of regions of interest in the image;
a step of performing determination of, with respect to each of the plurality of regions of interest, whether or not correction of data using the region of interest is possible; and
a step of correcting the data using the region of interest for which it is determined that the correction of the data is possible.

23. A non-transitory computer readable medium for storing a computer-executable program for causing a computer to function as an endoscope system that calculates oxygen saturation of an observation target, the computer-executable program causing the computer to realize:
a function of acquiring an image obtained by imaging the observation target;
a function of setting a plurality of regions of interest in the image;
a function of performing determination of, with respect to each of the plurality of regions of interest, whether or not correction of data using the region of interest is possible; and
a function of correcting the data using the region of interest for which it is determined that the correction of the data is possible.
